(19) United States
(12) Patent Application Publication
(10) Pub. No.: US 2025/0262414 A1
(43) Pub. Date: Aug. 21, 2025

(54) NEEDLE CAPTURE SAFETY INTERLOCK FOR CATHETER

(71) Applicant: Becton, Dickinson and Company, Franklin Lakes, NJ (US)

(72) Inventors: Siddarth SHEVGOOR, Mission Viejo, CA (US); John STOKES, Pleasant View, UT (US); Weston HARDING, Lehi, UT (US)

(73) Assignee: Becton, Dickinson and Company, Franklin Lakes, NJ (US)

(21) Appl. No.: 19/203,865

(22) Filed: May 9, 2025

Related U.S. Application Data
(63) Continuation-in-part of application No. 18/668,582, filed on May 20, 2024.
Application No. 18/668,582 is a continuation of application No. 18/109,118, filed on Feb. 13, 2023, now Pat. No. 12,023,455.
Application No. 18/109,118 is a continuation of application No. 16/995,699, filed on Aug. 17, 2020, now Pat. No. 11,607,530.
Application No. 16/995,699 is a continuation of application No. 15/304,375, filed on Oct. 14, 2016, now Pat. No. 10,780,249.
(60) Provisional application No. 62/077,760, filed on Nov. 10, 2014.
(60) Provisional application No. 61/981,223, filed on Apr. 18, 2014.
(60) Provisional application No. 61/981,312, filed on Apr. 18, 2014.

(51) Int. Cl. A61M 25/06; A61M 5/158; A61M 5/32; A61M 5/34; A61M 25/00; A61M 39/06; A61M 39/22; A61M 39/24

(52) U.S. Cl. CPC A61M 25/0618; A61M 5/158; A61M 5/3202; A61M 5/3213; A61M 5/3232; A61M 5/34; A61M 25/0097; A61M 25/0606; A61M 25/0631; A61M 39/22; A61M 39/24; A61M 5/3273; A61M 2039/064; A61M 2039/066; A61M 2039/0673; A61M 2039/226

(57) ABSTRACT

A medical device, comprising a hub or housing including a push tab disposed on an upper surface of the hub or housing and a rib disposed on the upper surface of the hub or housing, the rib including a top surface and the rib being proximal to the push tab, and a cannula directly or indirectly connected to the hub or housing, wherein the top surface of the rib includes a cradle shape for resisting rotation of the hub or housing when contacted by a finger of a user.

### RELATED APPLICATIONS

This application is a continuation in part of U.S. patent application Ser. No. 18/668,582 filed on May 20, 2024, which is continuation of U.S. patent application Ser. No. 18/109,118 filed on Feb. 13, 2023 (issued as U.S. Pat. No. 12,023,455), which is a continuation of U.S. patent application Ser. No. 16/995,699 filed on Aug. 17, 2020 (issued as U.S. Pat. No. 11,607,530), which is a continuation of U.S. patent application Ser. No. 15/304,375 filed on Oct. 14, 2016 (issued as U.S. Pat. No. 10,780,249), which is a U.S. national stage application under 35 U.S.C. § 371 of International Application No. PCT/US2015/026542, filed on Apr. 17, 2015, which claims the benefit under 35 U.S.C. § 119(e) of U.S. Provisional Patent Application Ser. 61/981,223, filed on Apr. 18, 2014, U.S. Provisional Application 61/981,312, filed on Apr. 18, 2014, and U.S. Provisional Patent Application Ser. No. 62/077,760, filed on Nov. 10, 2014. Each of the above applications is hereby incorporated by reference in its entirety.

### FIELD

Various exemplary embodiments of the invention relate to catheters. Specifically, The present invention relates, in general, to an anti-rotation push tab for a medical device. More specifically, the anti-rotation push tab is especially adapted for use with intravenous catheters, as well as catheter introducers and guidewire introducers.

### BACKGROUND

Catheter assemblies are used to place a catheter properly into the vascular system of a patient. Once in place, catheters such as intravenous catheters may be used to infuse fluids including normal saline, medicinal compounds, and/or nutritional compositions into a patient in need of such treatment. Catheters additionally enable the removal of fluids from the circulatory system and monitoring of conditions within the vascular system of the patient.

An intravenous (IV) catheter is typically mounted over an introducer needle having a sharp distal tip in order to properly insert an IV catheter into a patient. At least the distal portion of the catheter tightly engages the outer surface of the needle to facilitate insertion of the catheter into the blood vessel. The distal tip of the needle preferably extends beyond the distal tip of the catheter.

Although typical IV catheter and introducer needle assemblies generally perform their functions satisfactorily, they do have certain drawbacks. Some PIVCs have issues with stability of the catheter hub when advancing it and are prone to free spinning on the insertion needle during the insertion process. Oftentimes, the catheter hub includes a push tab to aid in advancing the catheter hub. As the catheter hub advances, in some cases, it experiences rolling where the catheter hub spins along the axis of the insertion needle. This can cause a problem when the push tab rotates out of reach of the finger being used to advance the catheter hub.

In some cases an edge is provided on the catheter hub so that the user can advance the catheter hub regardless of its angular position. There is a concern in that the edge becomes quite uncomfortable to a patient when the catheter hub has been taped down at the insertion site thus forcing the edge against the patient's soft tissue.

### SUMMARY OF THE INVENTION

It is an aspect of the present invention to provide a catheter assembly in which an improved clip and needle shield are used for needle protection. The improved arrangement is more compact, provides increased needle protection, and reduces the size and complexity of the catheter assembly. The addition of a release notch in a collar of a catheter hub and disengagement of the clip via the notch allows the needle shield to be more compact than in the prior art. In the prior art, without the notch, the clip has to travel a longer distance to disengage the catheter hub. In addition, the width of the needle shield is reduced by an improved attachment interface between the clip and the needle shield. Specifically, a spade attaches the clip to the needle shield with an outer surface of the spade exposed to an outside of the needle shield.

It is another an aspect of the present invention to provide features that oppose the rotational movement of a medical device in relation to the user's finger. In the case of an IV catheter, this can enhance the stability of the catheter during insertion, hooding, and threading. Embodiments of the present invention provide a platform that pushes on the user's finger when the catheter begins to rotate and allows the user's finger to resist the rotation and also steer the catheter back to the neutral starting position. Free spinning of the catheter hub can be prevented without making any other design compromises or increasing the cost of the design.

The foregoing and/or other aspects of the present invention can be achieved by providing a catheter assembly comprising a catheter, a needle having a sharp distal tip disposed in the catheter, a catheter hub housing the catheter and the needle, the catheter hub having a notch, a needle shield connected to the catheter hub when the needle is in a first position, and a clip disposed in the needle shield that cooperates with the needle, wherein the clip engages the collar in the first position of the needle, the clip disengages the collar via the notch when the needle is retracted to a second position to enclose at least a portion of the needle.

The foregoing and/or other aspects of the present invention can be achieved by also providing a catheter assembly comprising a catheter, a needle having a sharp distal tip disposed in the catheter, a catheter hub housing the catheter and the needle, a needle shield configured to be connected to the catheter hub, and a clip disposed in the needle shield that cooperates with the needle, the clip including a spade that attaches the clip to the needle shield, wherein an outer surface of the spade is exposed to an outside of the catheter assembly.

The foregoing and/or other aspects of the present invention are further achieved by a medical device, comprising a hub or housing having a push tab including a main portion extending radially from an upper surface of the hub or housing, and at least one anti-rotation feature for resisting rotation of the hub or housing. A cannula is directly or indirectly connected to the hub or housing. The medical device may be a catheter, the cannula may be a catheter tube, and the hub or housing may be a catheter hub or an introducer needle tip shield for the catheter.

Furthermore, the foregoing and/or other aspects of the present invention are achieved by a medical device, comprising a housing having a push tab including a main portion extending radially from an upper surface of the housing, and at least one anti-rotation feature for resisting rotation of the housing, and a cannula connected to the housing.

The foregoing and/or other aspects of the present invention can be achieved by further providing a method of operating a catheter assembly comprising disposing a needle having a sharp distal tip in a catheter, biasing a clip when the needle is in use in a first position, removing the needle from a catheter hub having a notch, releasing the clip when the needle is in a second position to enclose at least a portion of the needle, and disengaging the clip from the collar via the notch when the needle is in the second position.

### BRIEF DESCRIPTION OF THE DRAWINGS

The above aspects and features of the present invention will be more apparent from the description for the exemplary embodiments of the present invention taken with reference to the accompanying drawings, in which:

FIG. 1 illustrates a top left perspective view of a catheter assembly in accordance with an embodiment of the present invention;

FIG. 2 illustrates a side left perspective view of a catheter assembly;

FIG. 3 illustrates an alternate side left perspective view of a catheter assembly;

FIG. 4 illustrates a top plan view of the catheter assembly;

FIG. 5 illustrates a right side elevation view of the catheter assembly;

FIG. 6 illustrates a bottom plan view of the catheter assembly;

FIG. 7 illustrates a left perspective view of the assembled catheter hub, needle shield, and needle of the catheter assembly;

FIG. 8 illustrates a right perspective view of the assembled catheter hub, needle shield, and needle of the catheter assembly;

FIG. 9 illustrates a right side elevation view of the assembled catheter hub, needle shield, and needle of the catheter assembly;

FIG. 10 illustrates a bottom plan view of the assembled catheter hub, needle shield, and needle of the catheter assembly;

FIG. 11 illustrates a bottom plan view of the assembled catheter hub, needle shield, and needle of the catheter assembly;

FIG. 12 illustrates a top plan view of the assembled catheter hub, needle shield, and needle of the catheter assembly;

FIG. 13 illustrates a cross sectional view of a right side elevation view of the assembled catheter hub, needle shield, and needle of the catheter assembly;

FIG. 14 illustrates a left perspective view of the separated catheter hub, needle shield, and needle of the catheter assembly;

FIG. 15 illustrates a right perspective view of the separated catheter hub, needle shield, and needle of the catheter assembly;

FIG. 16 illustrates a right side elevation view of the separated catheter hub, needle shield, and needle of the catheter assembly;

FIG. 17 illustrates a second right side elevation view of the separated catheter hub, needle shield, and needle of the catheter assembly;

FIG. 18 illustrates a bottom plan view of the separated catheter hub, needle shield, and needle of the catheter assembly;

FIG. 19 illustrates a top plan view of the separated catheter hub, needle shield, and needle of the catheter assembly;

FIG. 20 illustrates a cross sectional view of a right side elevation view of the separated catheter hub, needle shield, and needle of the catheter assembly;

FIG. 21 is a right perspective view of the catheter hub of the catheter assembly;

FIG. 22 is a top plan view of the catheter hub of the catheter assembly;

FIG. 23 is a left perspective view of the catheter hub of the catheter assembly;

FIG. 24 is a front side view of the catheter hub of the catheter assembly;

FIG. 25 is a right side view of the catheter hub of the catheter assembly;

FIG. 26 is a rear side view of the catheter hub of the catheter assembly;

FIG. 27 is a left perspective view of the catheter hub of the catheter assembly;

FIG. 28 is a bottom plan view of the catheter hub of the catheter assembly;

FIG. 29 is a right perspective view of the catheter hub of the catheter assembly;

FIG. 30 illustrates a right perspective view of the needle shield outer housing of the catheter assembly;

FIG. 31 illustrates a left perspective view of the needle shield outer housing of the catheter assembly;

FIG. 32 illustrates a second left perspective view of the needle shield outer housing of the catheter assembly;

FIG. 33 illustrates a second right perspective view of the needle shield outer housing of the catheter assembly;

FIG. 34 illustrates a front side elevation view of the needle shield outer housing of the catheter assembly;

FIG. 35 illustrates a rear side elevation view of the needle shield outer housing of the catheter assembly;

FIG. 36 illustrates a right side elevation view of the needle shield outer housing of the catheter assembly;

FIG. 37 illustrates a right side elevation view of the needle shield outer housing of the catheter assembly;

FIG. 38 illustrates a top plan view of the needle shield outer housing of the catheter assembly;

FIG. 39 illustrates a bottom plan view of the needle shield outer housing of the catheter assembly;

FIG. 40 illustrates a left perspective view of the V-shaped metal clip of the catheter assembly;

FIG. 41 illustrates a right perspective view of the V-shaped metal clip of the catheter assembly;

FIG. 42 illustrates a second right perspective view of the V-shaped metal clip of the catheter assembly;

FIG. 43 illustrates a front elevation view of the V-shaped metal clip of the catheter assembly;

FIG. 44 illustrates a rear elevation view of the V-shaped metal clip of the catheter assembly;

FIG. 45 illustrates a left side elevation view of the V-shaped metal clip of the catheter assembly;

FIG. 46 illustrates a right side elevation view of the V-shaped metal clip of the catheter assembly;

FIG. 47 illustrates a top plan view of the V-shaped metal clip of the catheter assembly;

FIG. 48 illustrates a bottom plan view of the V-shaped metal clip of the catheter assembly;

FIG. 49 illustrates a right perspective view of the washer of the catheter assembly;

FIG. 50 illustrates a left perspective view of the washer of the catheter assembly;

FIG. 51 illustrates a front elevation view of the washer of the catheter assembly;

FIG. 52 illustrates a bottom plan view of the washer of the catheter assembly;

FIG. 53 illustrates a cross sectional view of a left side elevation view of the washer of the catheter assembly;

FIG. 54 illustrates a right perspective view of an alternative catheter hub with wings;

FIG. 55 illustrates a top plan view of the catheter hub with wings;

FIG. 56 illustrates a left perspective view of the catheter hub with wings;

FIG. 57 illustrates a front view of the catheter hub with wings;

FIG. 58 illustrates a left side elevation view of the catheter hub with wings;

FIG. 59 illustrates a rear elevation view of the catheter hub with wings;

FIG. 60 illustrates a left perspective view of the catheter hub with wings;

FIG. 61 illustrates a bottom plan view of the catheter hub with wings;

FIG. 62 illustrates a second right perspective view of the catheter hub with wings;

FIG. 63 illustrates a right perspective view of an alternative side port catheter hub;

FIG. 64 illustrates a top plan view of the side port catheter hub;

FIG. 65 illustrates a left perspective view of the side port catheter hub;

FIG. 66 illustrates a front elevation view of the side port catheter hub;

FIG. 67 illustrates a right side elevation view of the side port catheter hub;

FIG. 68 illustrates a rear elevation view of the side port catheter hub;

FIG. 69 illustrates a second left perspective view of the side port catheter hub;

FIG. 70 illustrates a bottom plan view of the side port catheter hub;

FIG. 71 illustrates a second right perspective view of the side port catheter hub;

FIG. 72 illustrates a left perspective view of the alternative side port catheter hub assembly with a needle shield and needle hub;

FIG. 73 illustrates a right perspective view of the alternative side port catheter hub assembly with a needle shield and needle hub;

FIG. 74 illustrates a top plan view of the alternative side port catheter hub assembly with a needle shield and needle hub;

FIG. 75 illustrates a right side view of the alternative side port catheter hub assembly with a needle shield and needle hub;

FIG. 76 illustrates a bottom plan view of the alternative side port catheter hub assembly with a needle shield and needle hub;

FIG. 77 illustrates a cross sectional view of the catheter assembly of FIGS. 1-12 as the introducer needle is being withdrawn;

FIG. 78 illustrates a second cross sectional view of the catheter assembly of FIGS. 1-12 as the introducer needle is being withdrawn;

FIG. 79 illustrates a cross sectional view of the catheter assembly of FIGS. 1-16 as the introducer needle is moved past the V-shaped metal clip and the needle shield is separated from the catheter hub;

FIG. 80 illustrates a second cross sectional view of the catheter assembly of FIGS. 1-16 as the introducer needle is moved past the V-shaped metal clip and the needle shield is separated from the catheter hub;

FIG. 81 illustrates a latch of the V-shaped metal clip engaged with the catheter hub

FIG. 82 illustrates the latch of the V-shaped metal clip disengaged from the catheter hub;

FIG. 83 illustrates the latch of the V-shaped metal clip disengaged from the catheter hub and separated;

FIG. 84 illustrates a view of the V-shaped metal clip blocking the needle;

FIG. 85 illustrates a view of the V-shaped metal clip in the closed position;

FIG. 86 illustrates a schematic view of the working envelope of the V-shaped metal clip and catheter hub collar without a notch;

FIG. 87 illustrates a schematic view of the working envelope of the V-shaped metal clip and catheter hub collar without the notch;

FIG. 88 illustrates a schematic view of the working envelope of the V-shaped metal clip and notched catheter hub collar;

FIG. 89 illustrates a schematic view of the working envelope of the V-shaped metal clip and notched catheter hub collar;

FIG. 90 illustrates the operation of the catheter hub valve actuator in a free state;

FIG. 91 illustrates the operation of the catheter hub valve actuator in a compressed state;

FIG. 92 illustrates the operation of a second embodiment of a catheter hub valve actuator in a free state;

FIG. 93 illustrates the operation of the second embodiment of the catheter hub valve actuator in the compressed state;

FIG. 94 illustrates another embodiment of the catheter hub valve actuator;

FIG. 95 illustrates an exemplary blood flashback feature of the catheter assembly;

FIG. 96 illustrates the needle of the blood flashback feature of the catheter assembly of FIG. 95;

FIG. 97 illustrates a second exemplary blood flashback feature of the catheter assembly;

FIG. 98 illustrates the second exemplary blood flashback feature of the catheter assembly of FIG. 97 with blood flashback in two places;

FIG. 99 illustrates a third exemplary blood flashback features of the catheter assembly with blood flashback in three places;

FIG. 100 illustrates a right side view of the exemplary embodiment of the actuator of FIG. 94;

FIG. 101A illustrates a cross sectional view of the actuator of FIG. 100 in a catheter hub assembly;

FIG. 101B illustrates the cross sectional view of the catheter hub assembly of FIG. 101A when penetrating a septum;

FIG. 101C illustrates a left perspective cross sectional view of the catheter hub assembly of FIG. 101A when penetrating a septum;

FIG. 102A illustrates a cross sectional view of another exemplary embodiment of a catheter hub assembly;

FIG. 102B illustrates the cross sectional view of the catheter hub assembly of FIG. 102A when penetrating a septum; and

FIG. 102C illustrates a left perspective cross sectional view of the catheter hub assembly of FIG. 102A when penetrating a septum.

FIG. 103 illustrates a catheter hub incorporating an anti-rotation push tab;

FIG. 104 illustrates a catheter hub incorporating a sculpted anti-rotation push tab;

FIG. 105 illustrates a catheter hub incorporating a sculpted anti-rotation push tab including ribs;

FIG. 106 illustrates a catheter hub incorporating an extended anti-rotation push tab;

FIG. 107 illustrates a catheter hub incorporating a deep sculpted anti-rotation push tab;

FIG. 108 illustrates a catheter hub incorporating an anti-rotation push tab according to another embodiment of the invention;

FIG. 109 illustrates a catheter hub incorporating an anti-rotation push tab according to still another embodiment of the invention; and

FIGS. 110 and 111 illustrate a catheter hub incorporating an anti-rotation push tab according to yet another embodiment of the invention.

### DETAILED DESCRIPTION OF EXEMPLARY EMBODIMENTS

The catheter assembly disclosed herein is an improvement over that disclosed in commonly owned U.S. Patent Application Publication No. 2014/0364809, which is incorporated herein by reference.

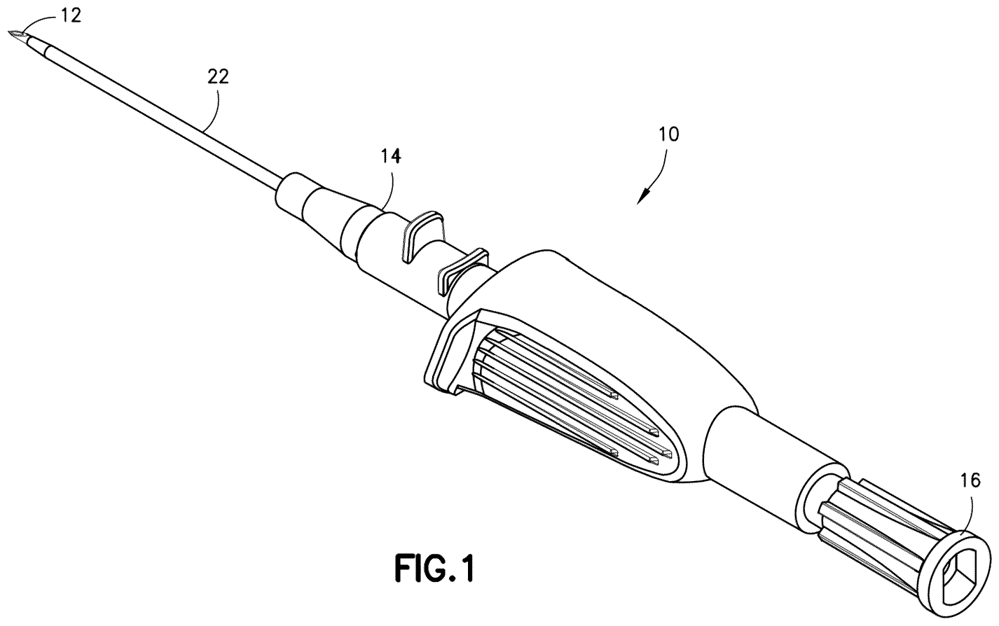

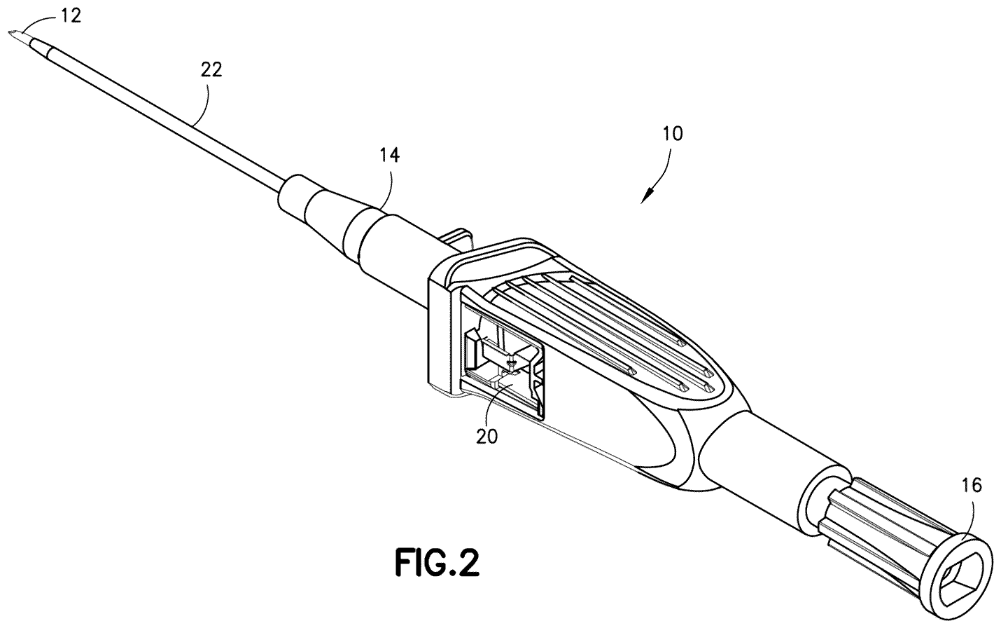

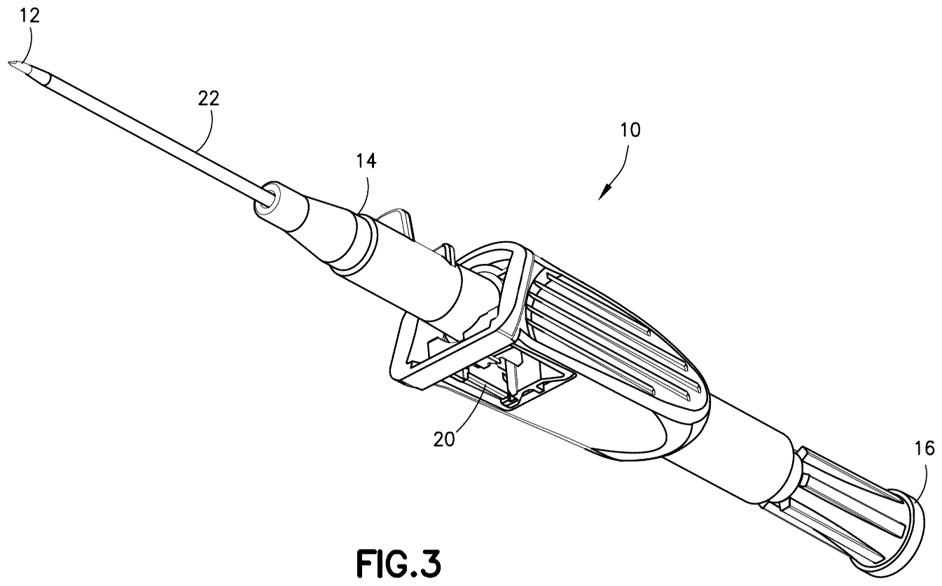

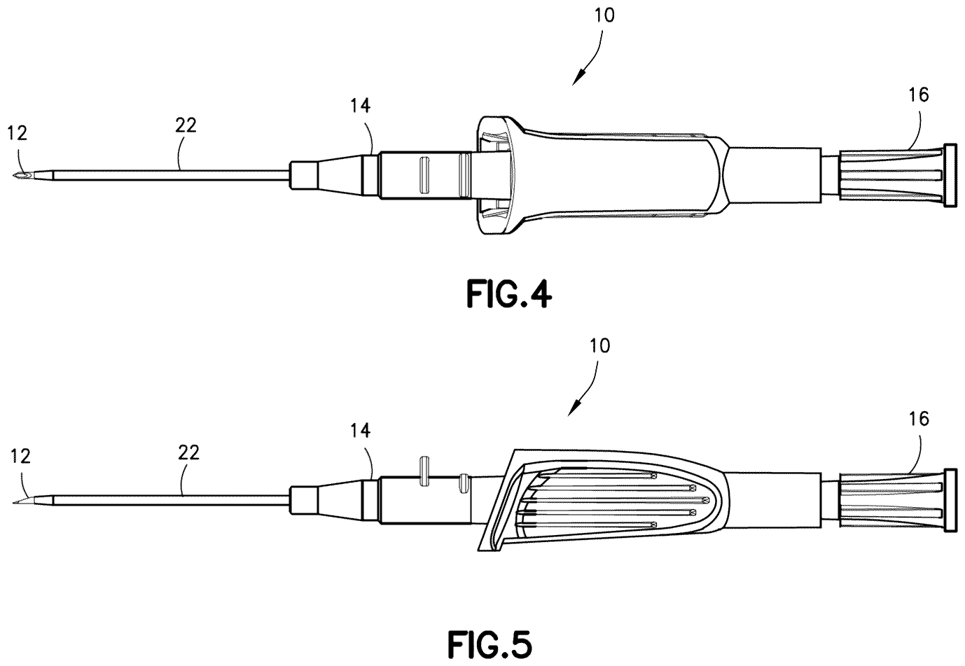

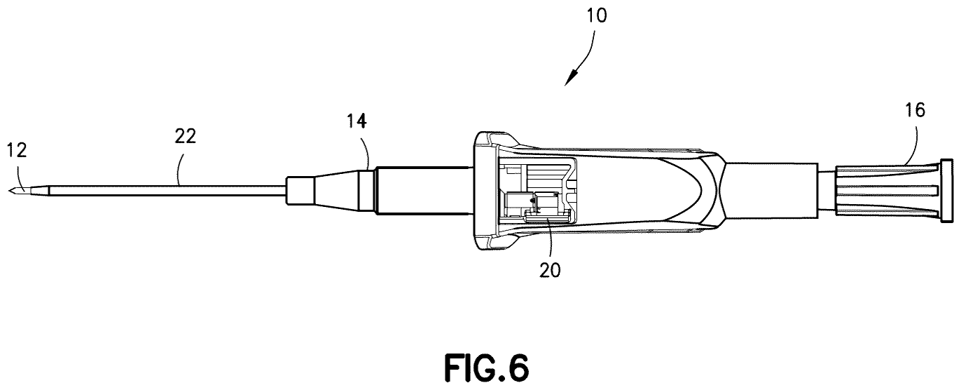

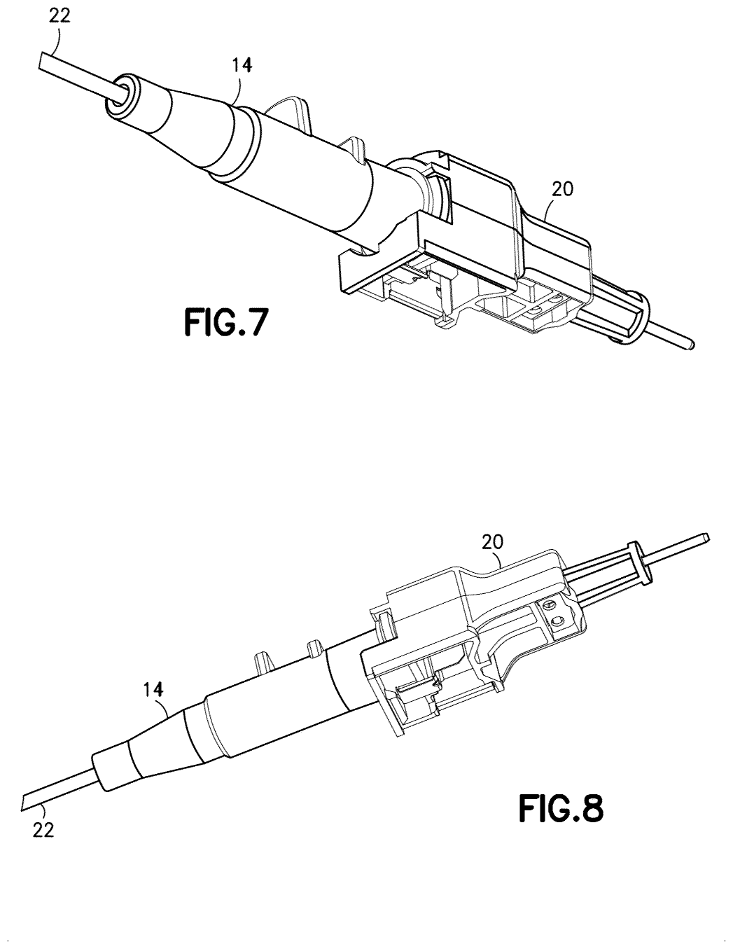

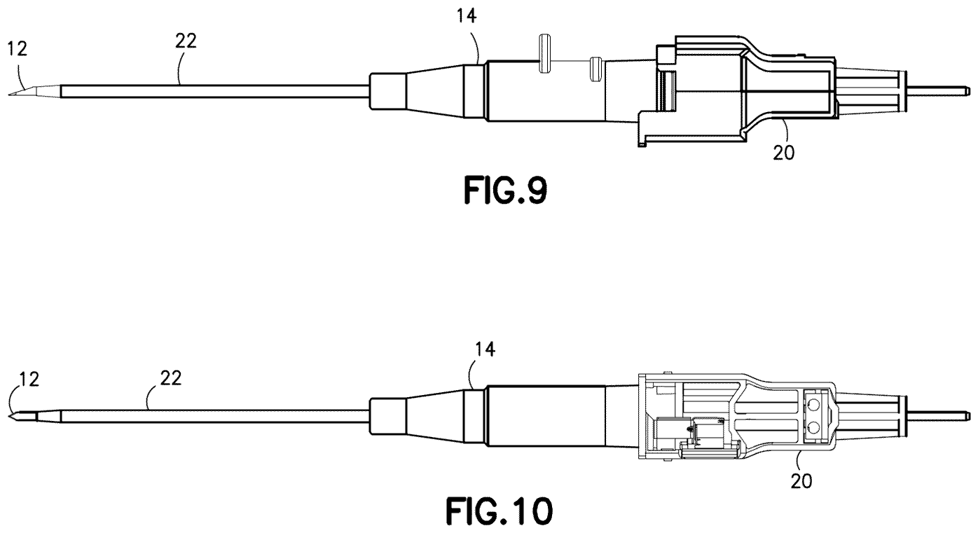

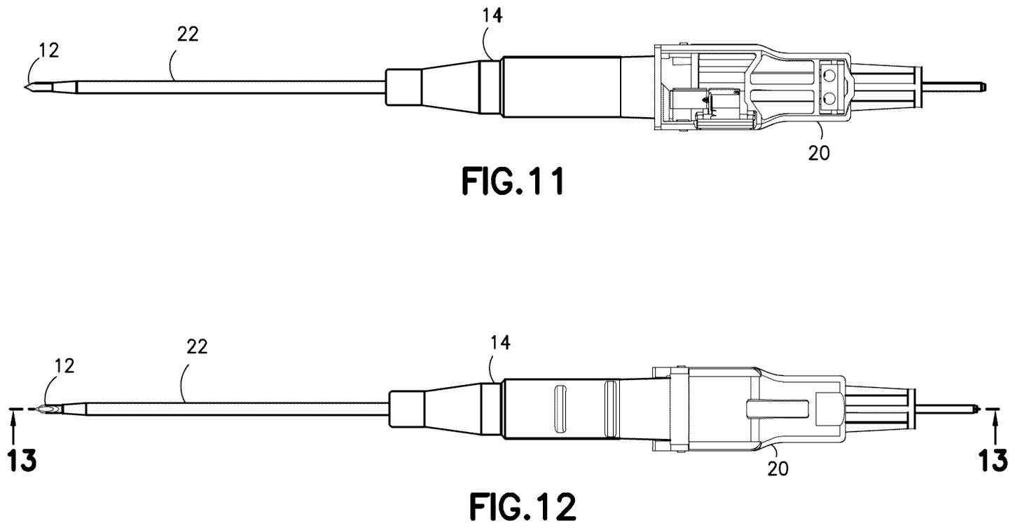

FIGS. 1-12 illustrate a catheter assembly 10 includes a hollow metal introducer needle 12, a catheter hub 14, a needle hub 16, and a needle shield 20. The needle 12 has a sharpened and beveled distal end and initially extends through the needle shield 20 and the catheter hub 14. A flexible catheter tube 22 extends from the distal end of the catheter hub 14, with the introducer needle 12 passing through the catheter tube 22. Initially, the needle 12 is inserted into a patient's vein. The catheter tube 22 is carried by the needle 12 into the vein. After the catheter tube 22 is inserted, the needle 12 is removed from the patient's vein and the catheter hub 14. The needle shield 20 encloses the tip of the needle 12 and provides protection from being stuck by the needle 12 during and after the needle's retraction from the catheter hub 14. The needle shield 20 can be used with a variety of different catheters.

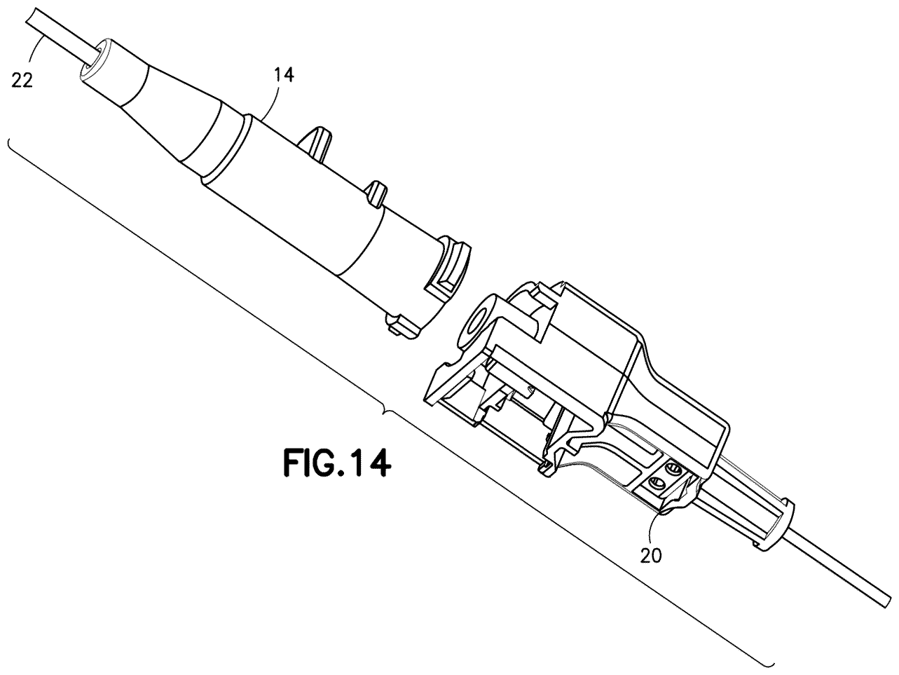

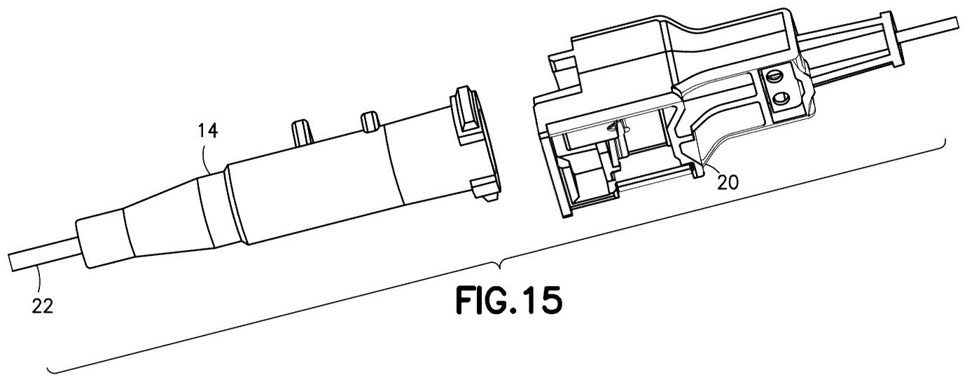

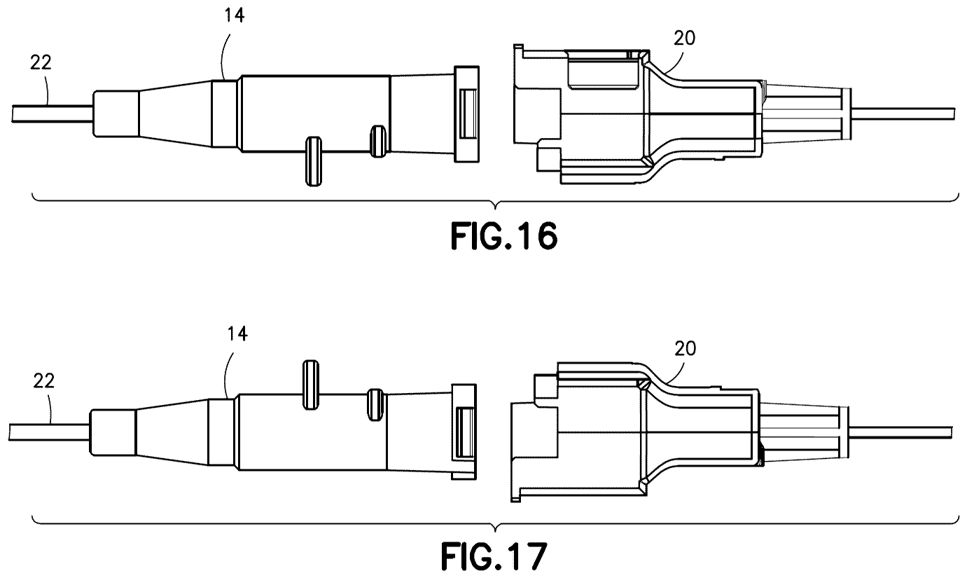

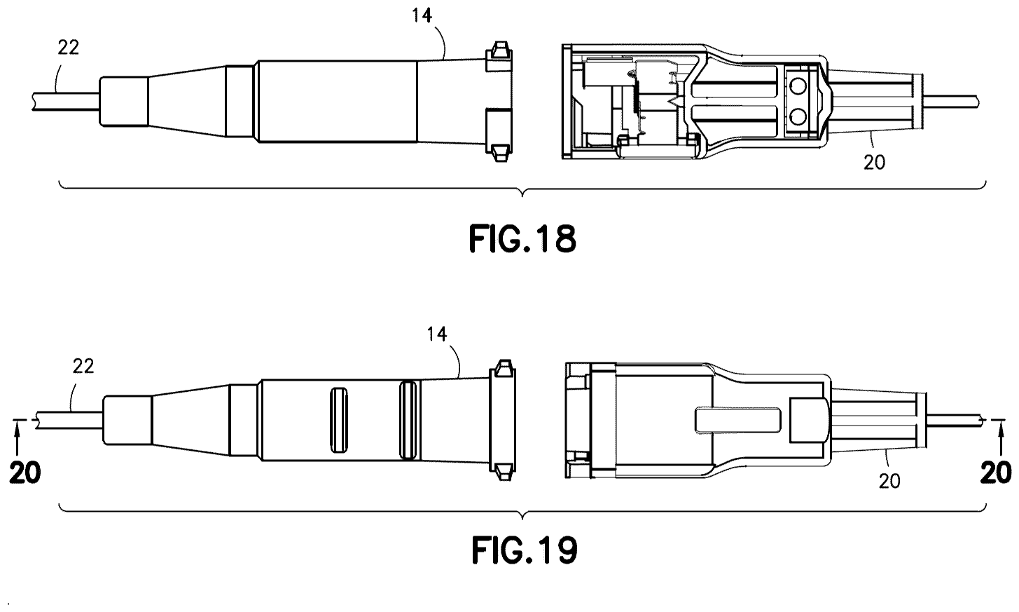

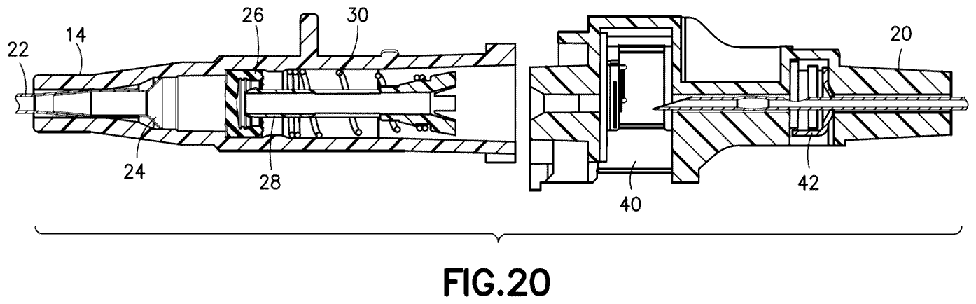

As illustrated in FIGS. 12-20, the catheter assembly includes the catheter hub 14 and the flexible catheter tube 22 extending from the catheter hub 14. A metal wedge 24 is positioned in the catheter hub 14 to retain the catheter tube 22. A resilient septum 26 is positioned to control fluid flow through the catheter hub 14. An actuator 28 is moveably positioned in the catheter hub 14 to engage the septum 26. A biasing member 30 engages the actuator 28 to bias the actuator 28 in the proximal direction.

The resilient septum 26 has one or more pre-formed slits which are normally closed to selectively prevent unwanted fluid flow through the septum 26. For example, the septum 26 can have three slits forming three triangular flaps that open when engaged by the actuator 28. The septum 26 is made from an elastic material, for example silicone rubber, that provides the resilient closing force for the slits. Other septum 26 configurations may be used as would be understood by one of ordinary skill in the art.

The actuator 28 and the biasing member 30, for example a metal or plastic compression spring, are positioned in the catheter hub 14. The actuator 28 engages the septum 26 to open the slits and permit fluid flow through the catheter hub 14. The biasing member 30 is capable of returning the actuator 28 to a position that allows the slits to close, preventing fluid flow through the catheter hub 14.

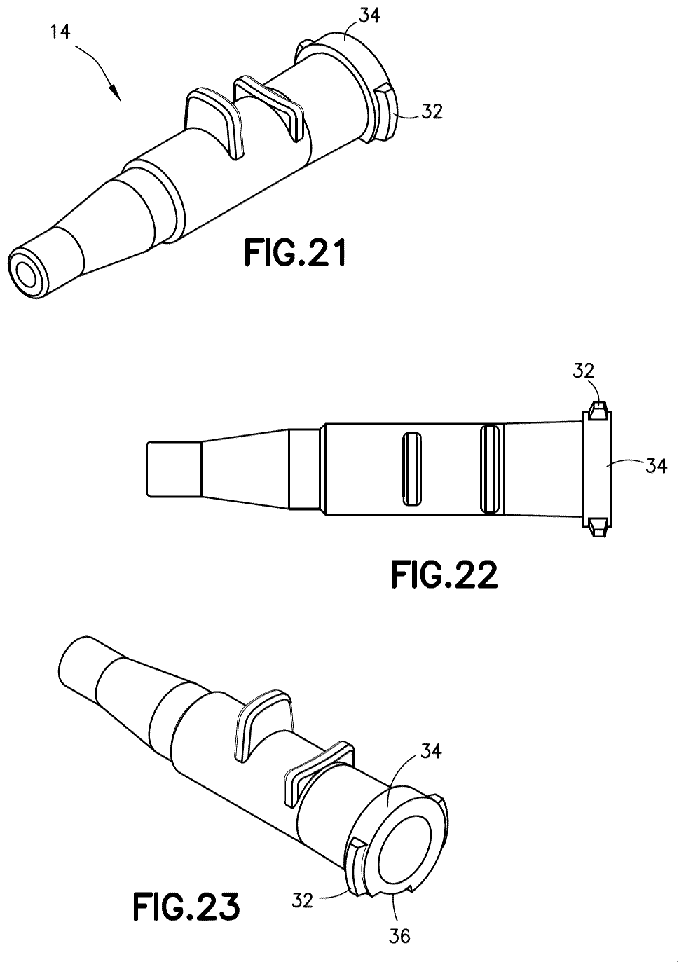

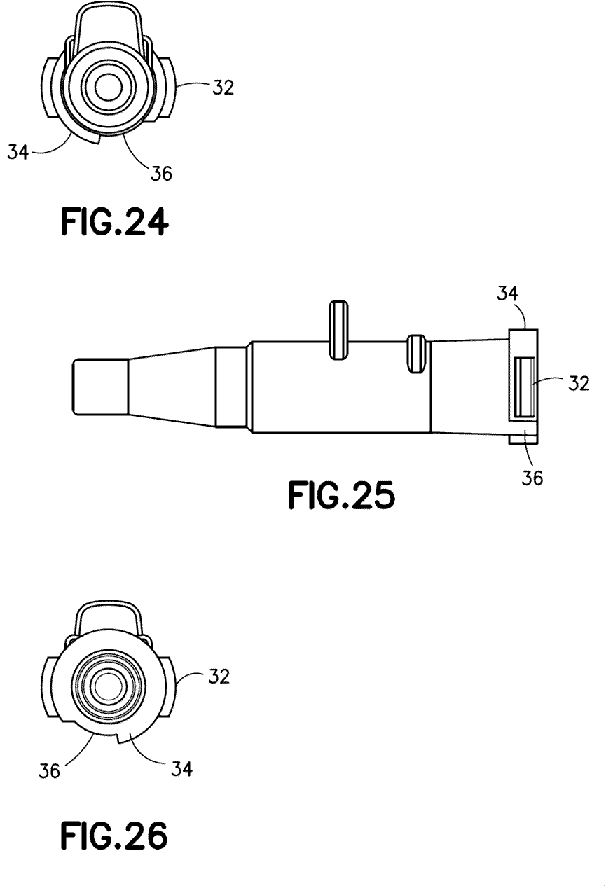

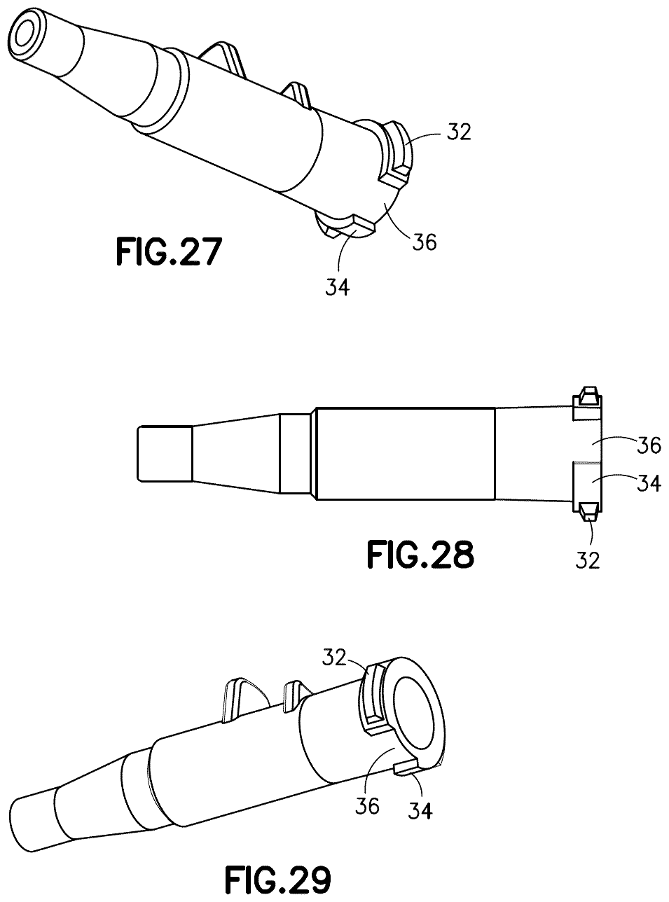

As best shown in the exemplary embodiment of FIGS. 21-29, the catheter hub 14 includes a proximal end having external Luer thread 32 and a notched collar 34. The collar 34 extends around at least a portion of the catheter hub 14 and is preferably disposed at a proximal end of the catheter hub 14. The collar 34 has a break, opening or notch 36 separating first and second ends of the collar 34.

A portion of the collar 34 includes an outer diameter that is greater than a portion of an outer diameter of the catheter hub 14 adjacent to the collar 34. Specifically, a portion of the outer diameter of the collar 34 is elevated with respect to the adjacent outer diameter surface of the catheter hub 14. Additionally, the opening 36 of the collar 34 has an outer diameter substantially equal to or greater than a portion of an outer diameter of the catheter hub 14 adjacent to the collar 34.

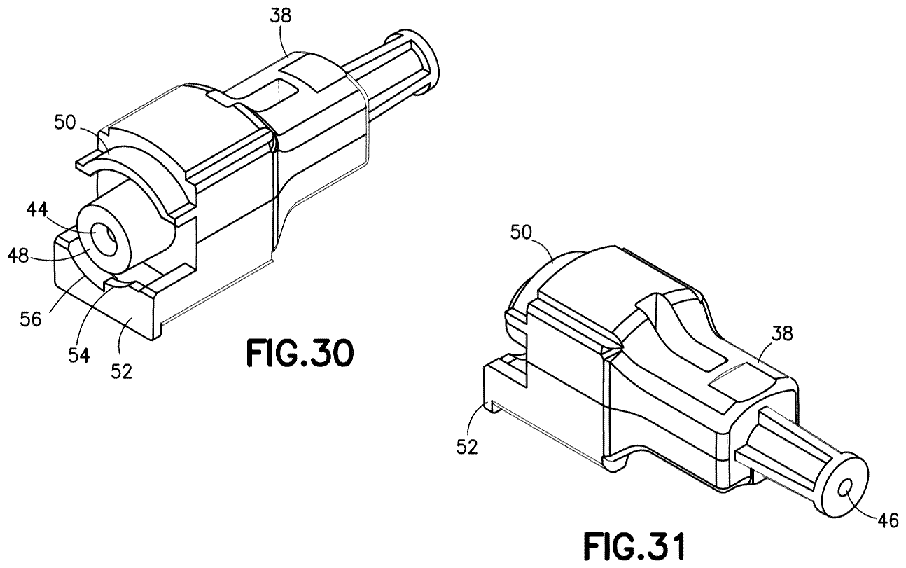

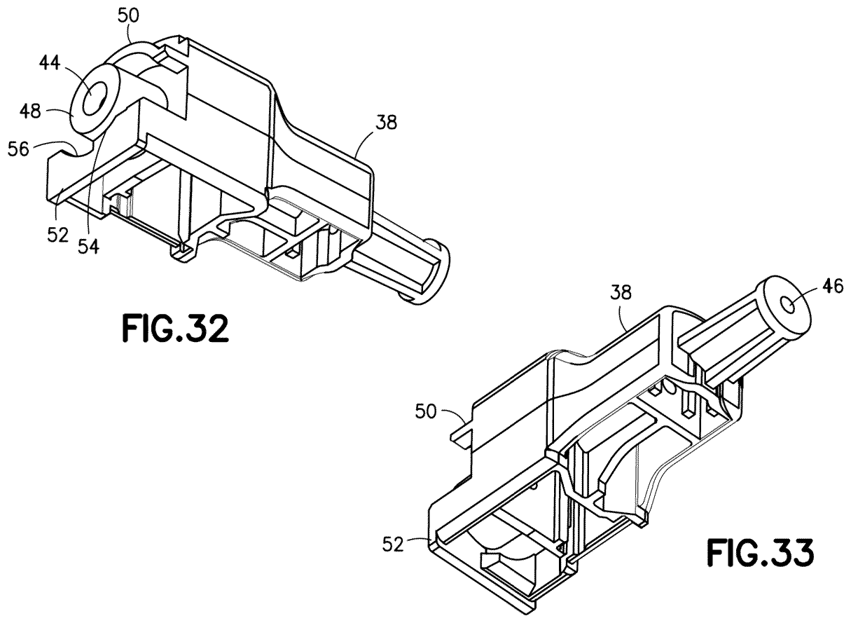

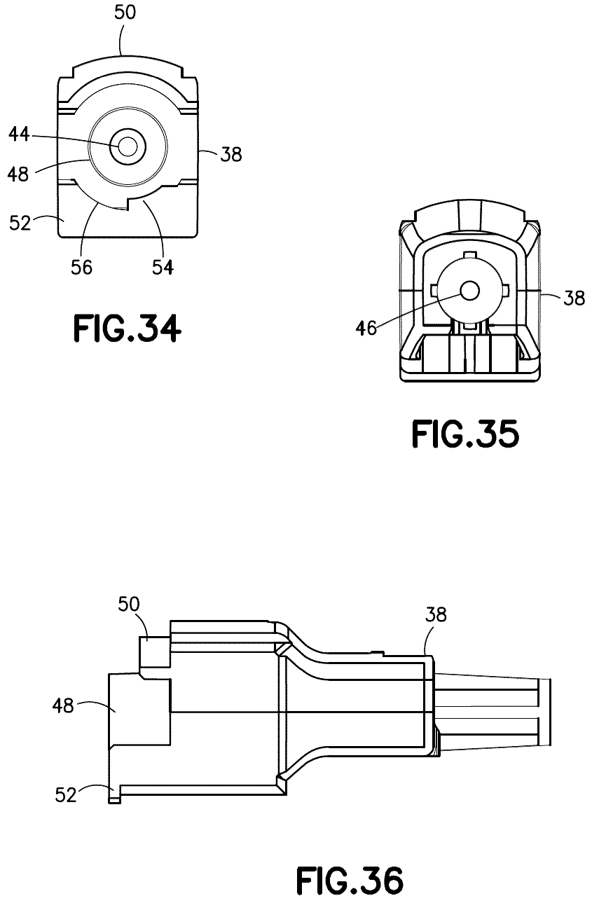

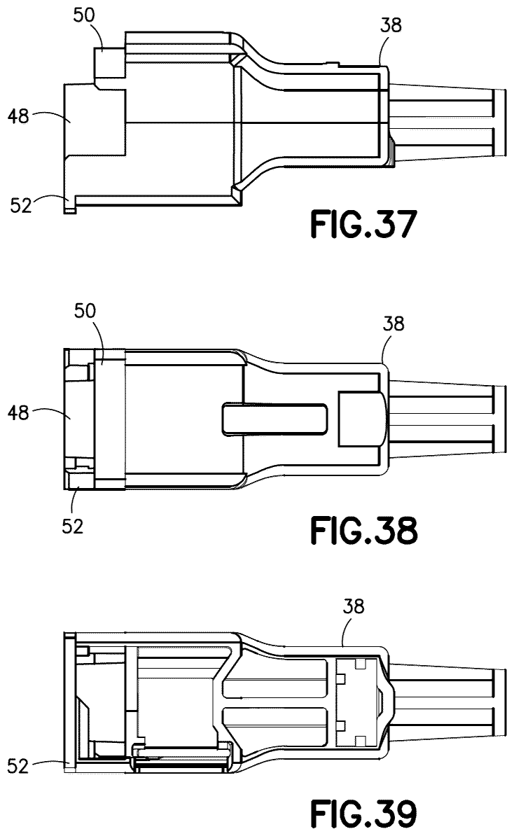

In an exemplary embodiment, the needle shield 20 includes an outer housing 38, a resilient clip 40, and a washer 42. The outer housing 38 includes an aperture having a distal opening 44 and a proximal opening 46 to receive the needle 12. The outer housing 38 connects to the catheter hub 14 and surrounds the clip 40 and the washer 42. As best shown in FIGS. 30-39, the distal end of the outer housing 38 includes a nose 48, a top flange 50, and a base 52. When the needle shield 20 is connected to the catheter hub 14, the nose 48 extends into the interior of the catheter hub 14.

In an exemplary embodiment, the nose 48 is sized to be slightly smaller than the interior of the catheter hub 14 so as to fit with a loose tolerance. The top flange 50 is spaced from the base 52 by a pair of side recesses that receive the Luer threads 32 and prevent rotation of the catheter hub 14 with respect to the needle shield 20 when assembled. The base 52 includes a projection 54 having a curved top surface and curved cut-out portion 56. The projection 54 is sized to fit in the opening 36 of the collar 34 and the cut-out portion 56 is sized to allow the collar 34 to pass therethrough.

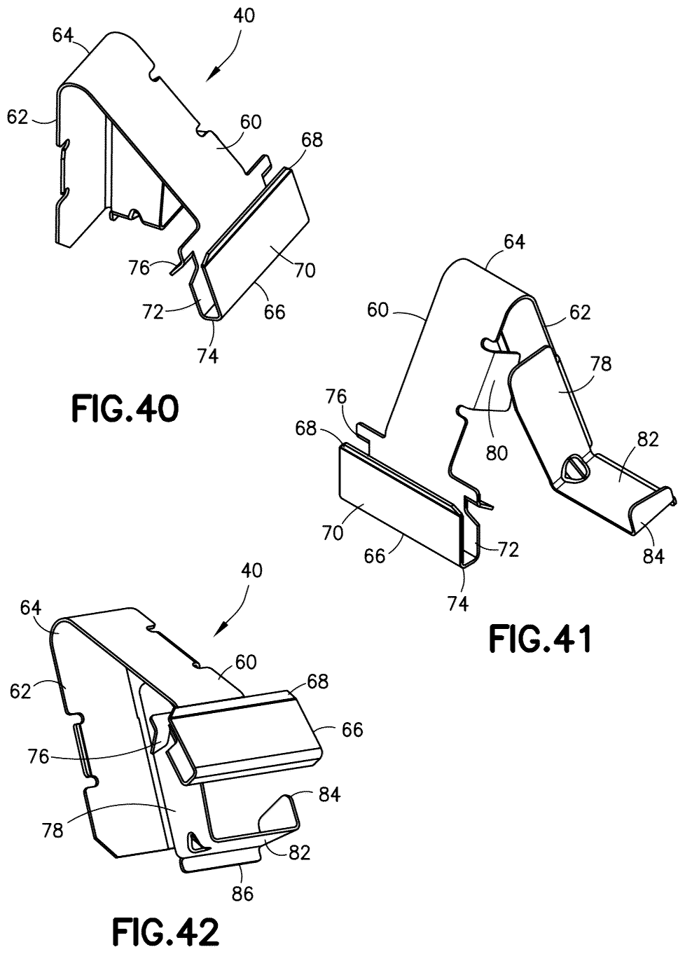

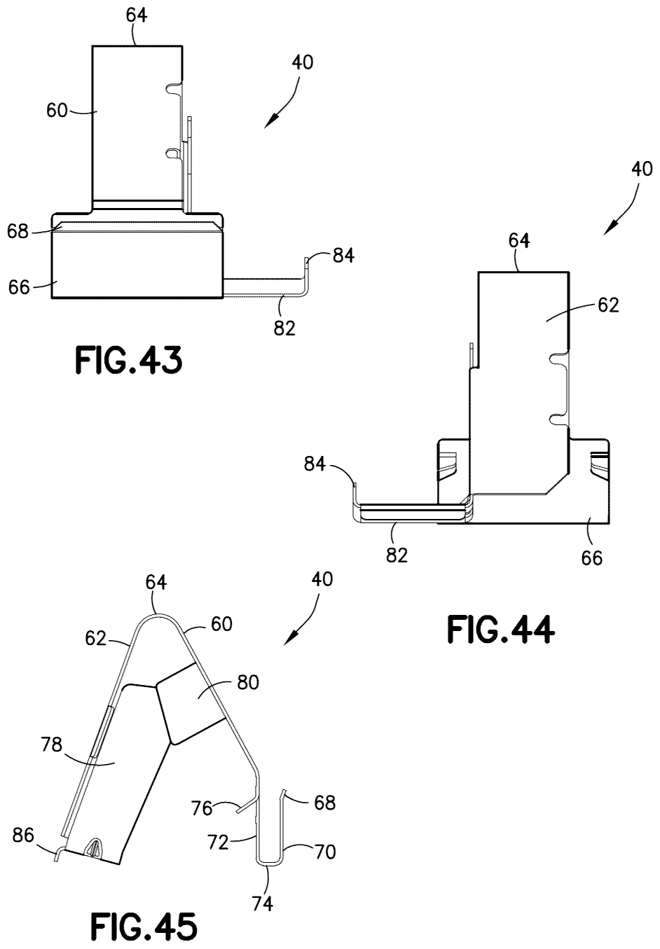

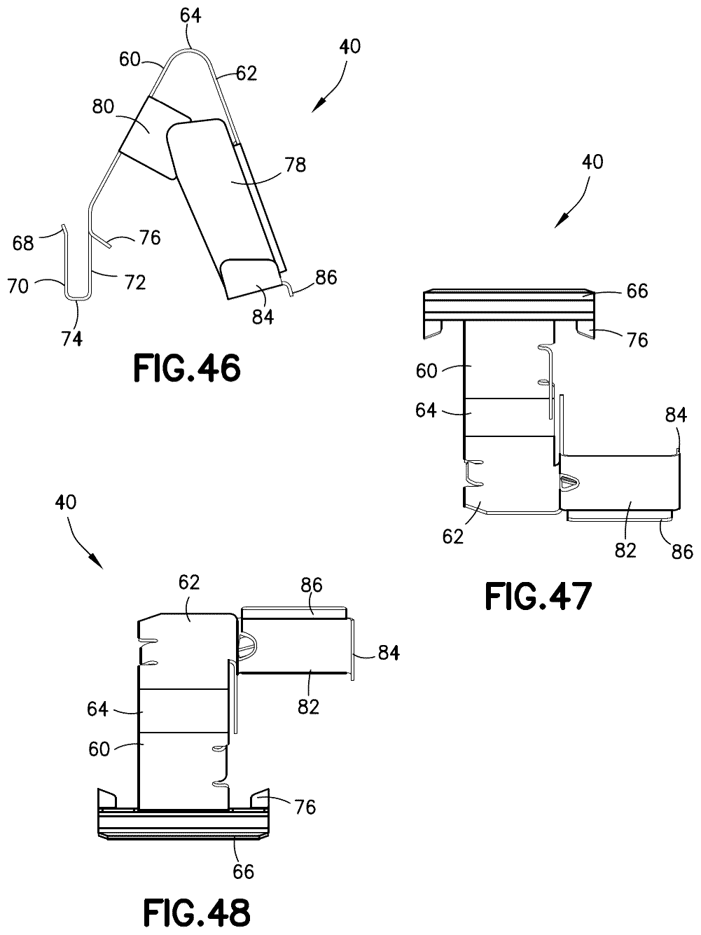

According to an exemplary embodiment illustrated in FIGS. 40-48, the clip 40 is a substantially V-shaped resilient clip 40 having a first leg 60 and a second leg 62 connected by an angled or curved V section 64. The first leg 60 includes a substantially U-shaped spade 66 having an angled lead-in portion 68. The spade 66 includes an outer wall 70 and an inner wall 72 connected by a bottom 74. A pair of barbs 76 extends outwardly from the inner wall 72 of the spade 66. A first flag 78 extends from the second leg 62 toward the first leg 60 and a second flag 80 extends from the first leg 60 toward the second leg 62. A foot 82 extends outwardly from the first flag 78 away from the first and second legs 60, 62, and a latch 84 extends upwardly from the foot 84 and is positioned between the first and second legs 60, 62. Specifically, the latch 84 is disposed between a plane representing the first leg 60 and a plane representing the second let 62. Such a configuration is desired to create a more compact clip 40. An optional stiffener 86 can extend downwardly from the foot 82.

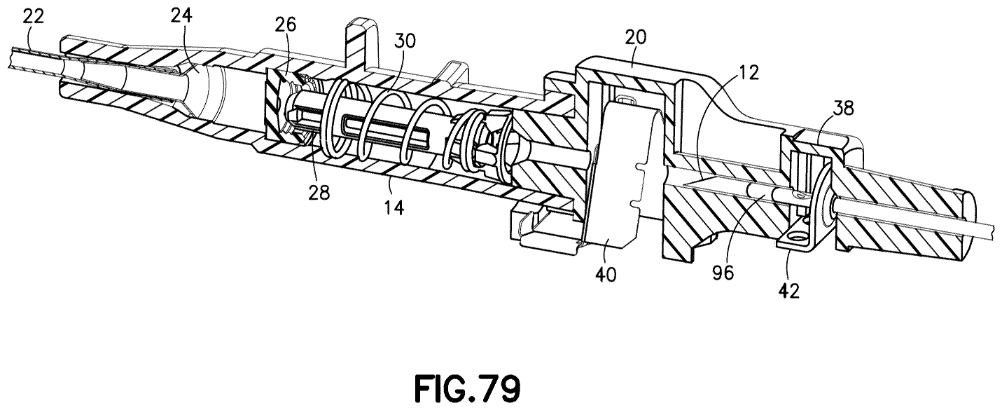

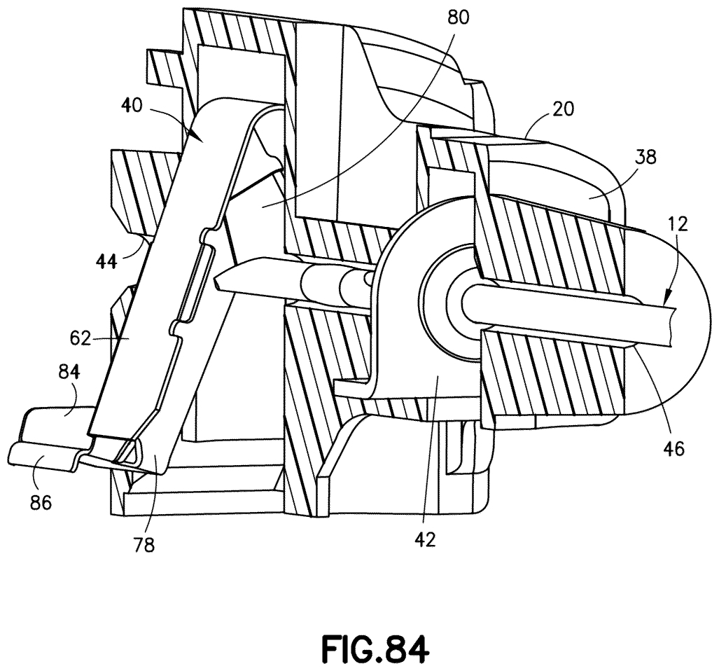

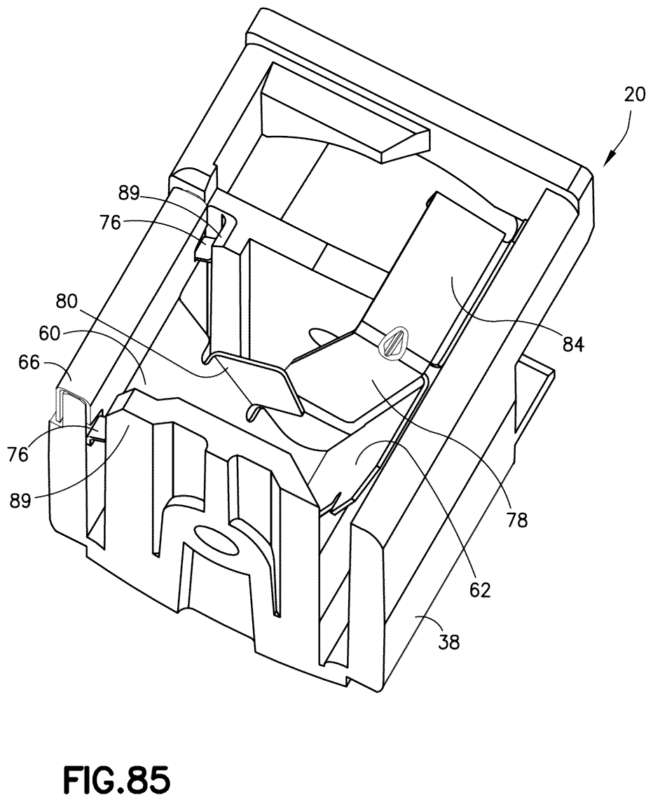

The clip 40 is connected to the outer housing 38 with the spade 66 being positioned around an exterior wall of the outer housing 38. The spade 66 is attached to the exterior wall of the outer housing 38 so that the outer wall 70 of the spade 66 is exposed to the outside of the needle shield 20. This configuration advantageously reduces the width of the needle shield 20, compared to an arrangement wherein the spade 66 is received within an internal cavity of the outer housing 38 such that the outer wall 70 is not exposed to the outside of the outer housing 38. As best shown in FIGS. 79-85, the inner wall 72 of the spade 66 is positioned in a recess and the two barbs 76 extend away from the inner wall 72 to engage a pair of projections 89 in the needle shield 20. The two barbs 76 aid in securely fastening the clip 40 to an inner surface of the needle shield 20. The clip 40 may be formed from a metal, elastomer, polymer, or composite material. In various exemplary embodiments, the clip 40 is formed from a thin piece of resilient metal, such as stainless steel.

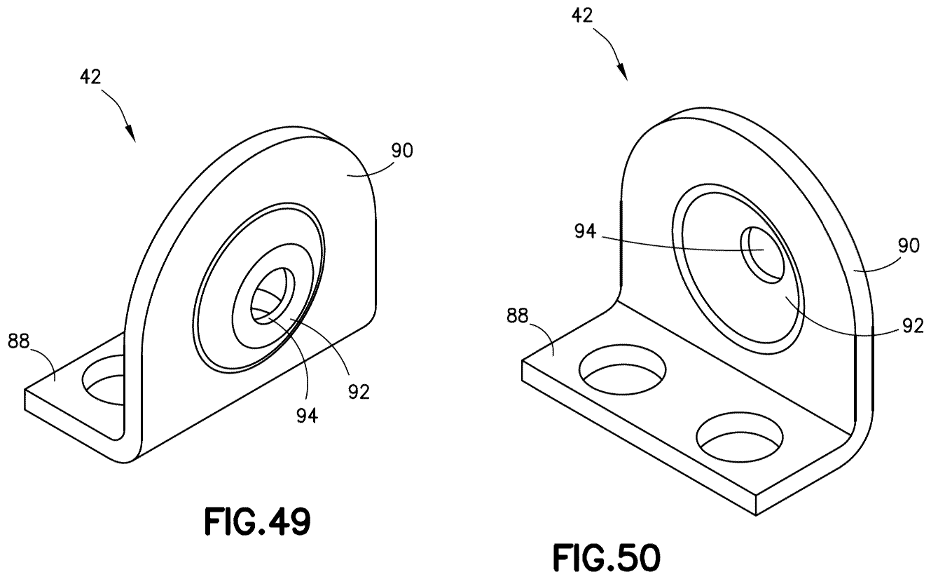

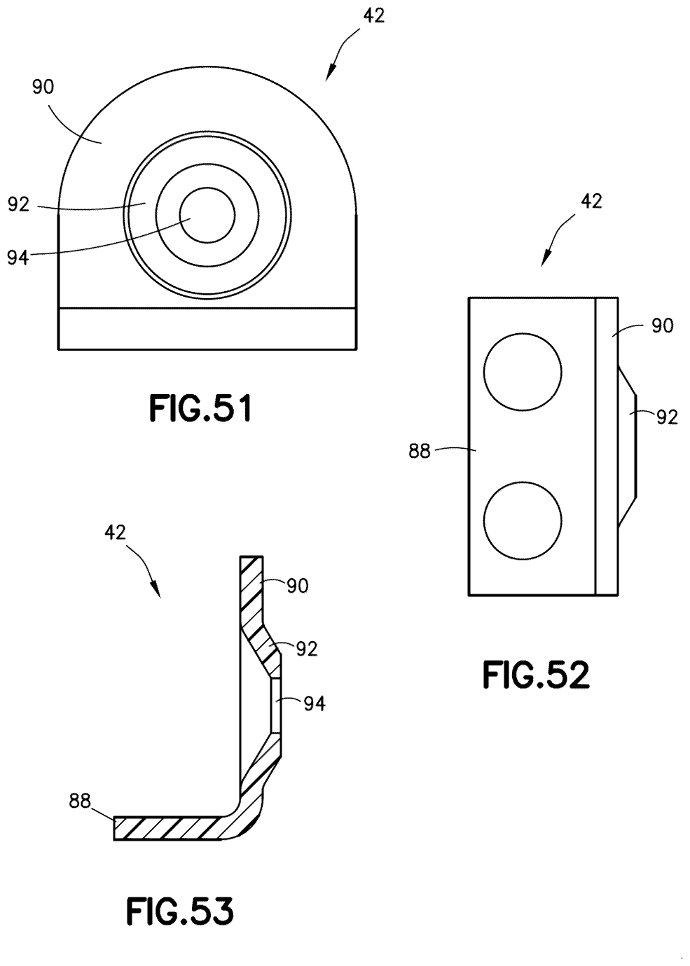

According to an exemplary embodiment illustrated in FIGS. 49-53, the washer 42 includes a base 88 and a side wall 90 connected together in a substantially L-shape. The side wall 90 includes a funnel 92 and an opening 94. The needle 12 includes a deformation 96, for example a crimp or protrusion formed near the distal end of the needle 12. The opening 94 in the washer 42 is sized to allow passage of the needle shaft, but not the deformation 96. The funnel 92 makes it easier for the proximal end of the needle 12 to be initially inserted through the washer 42 during assembly.

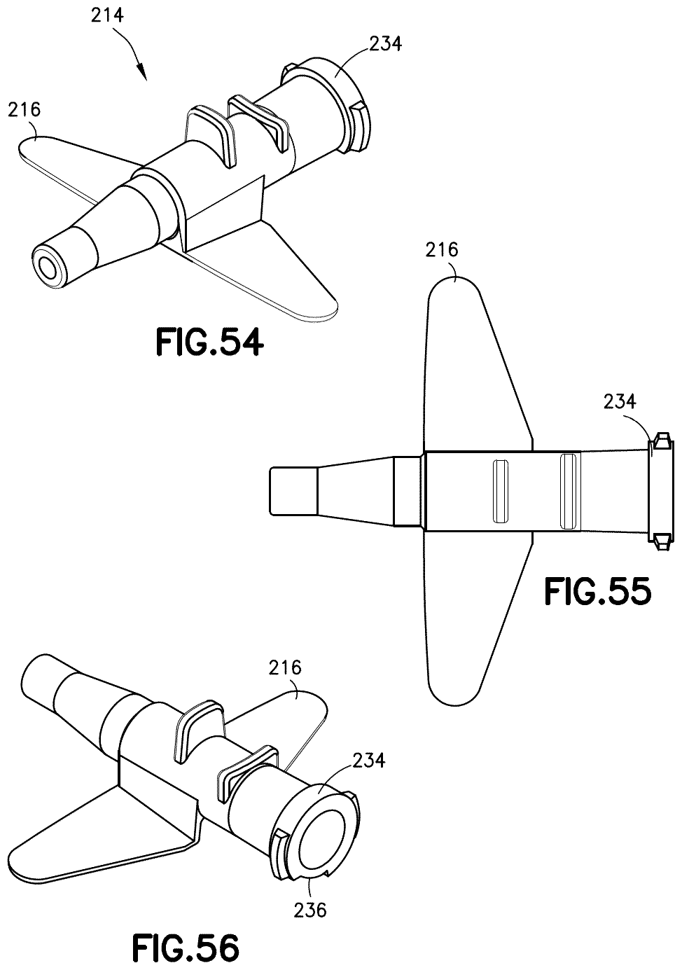

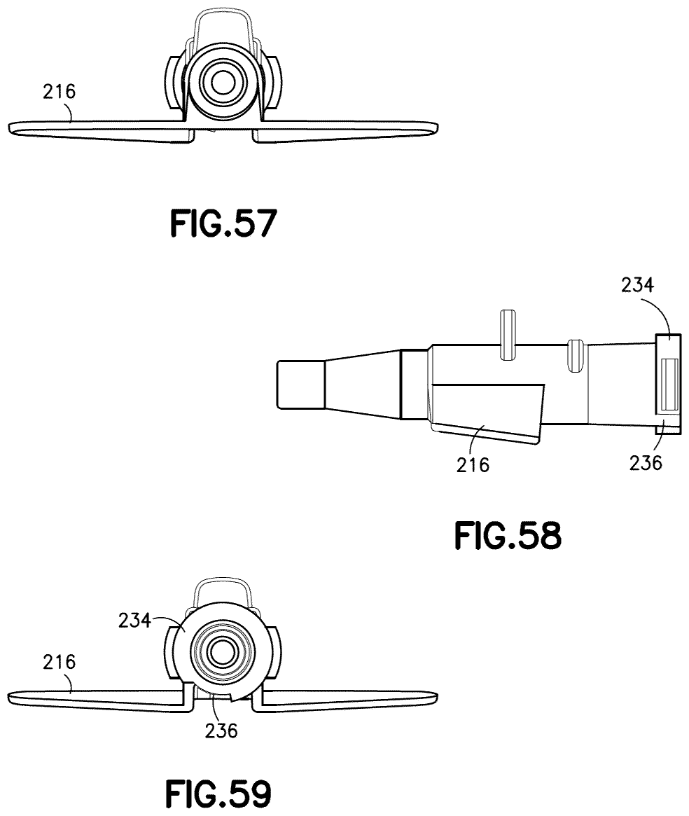

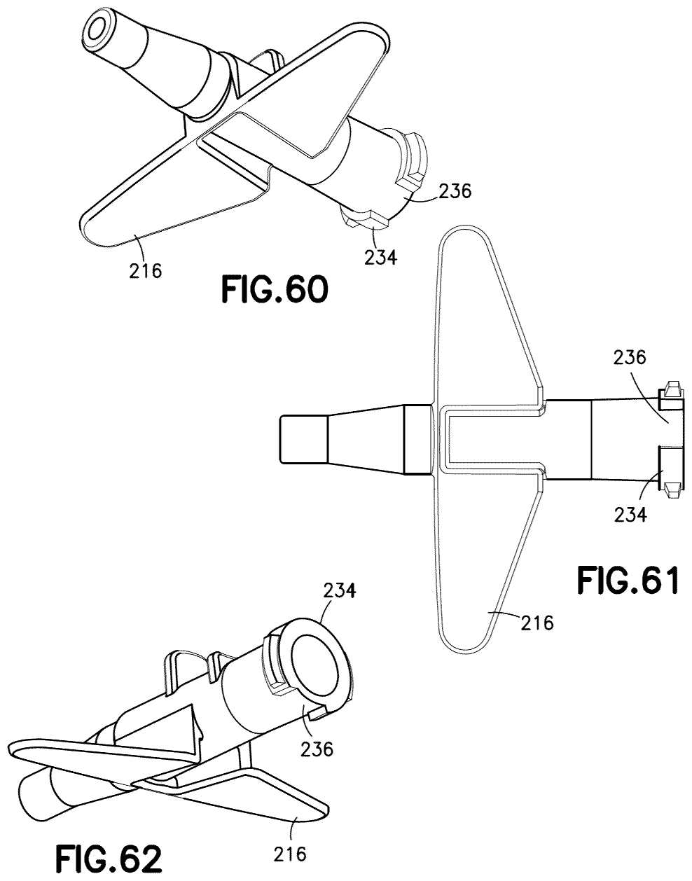

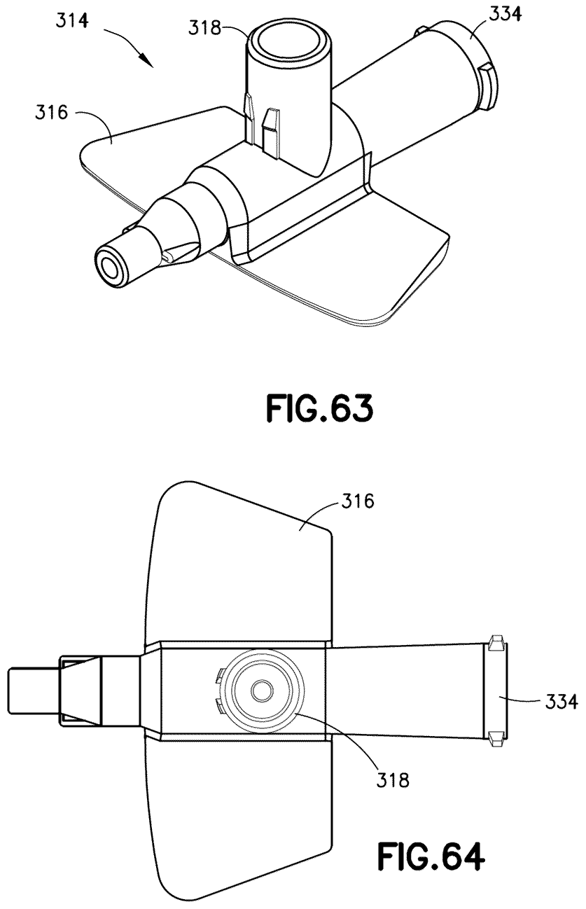

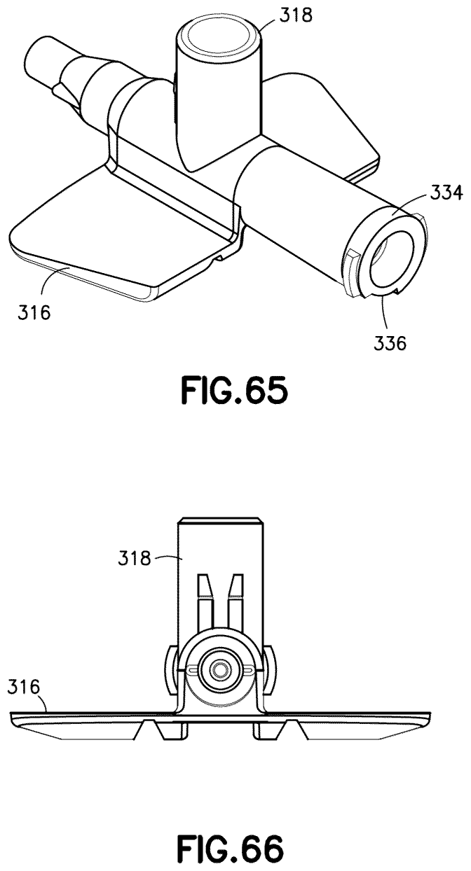

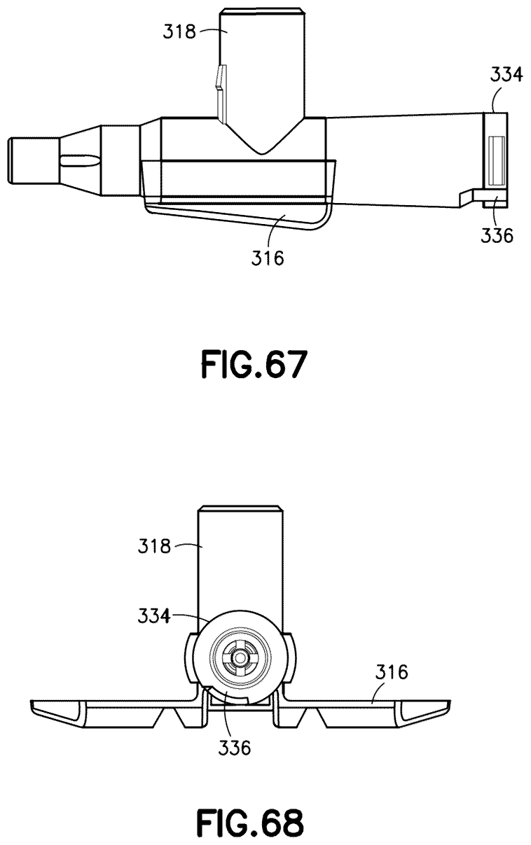

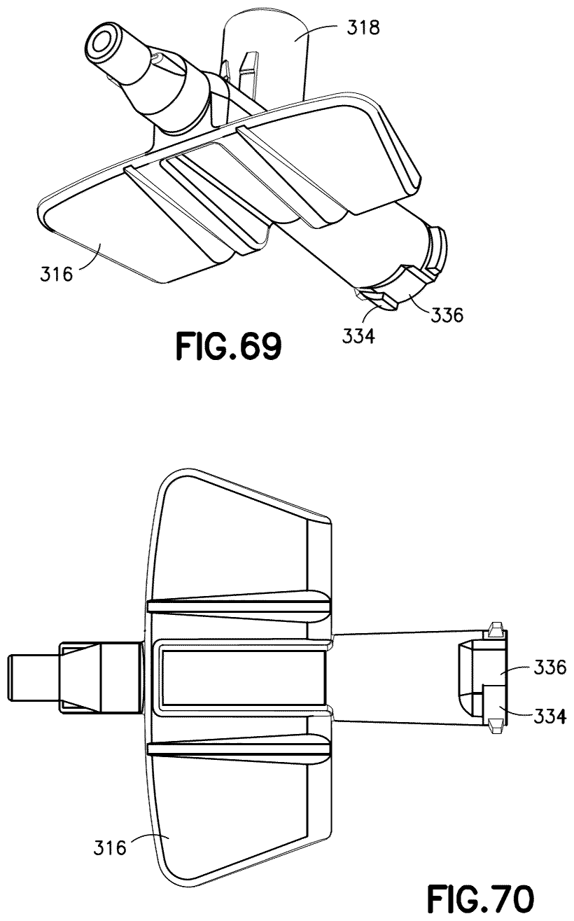

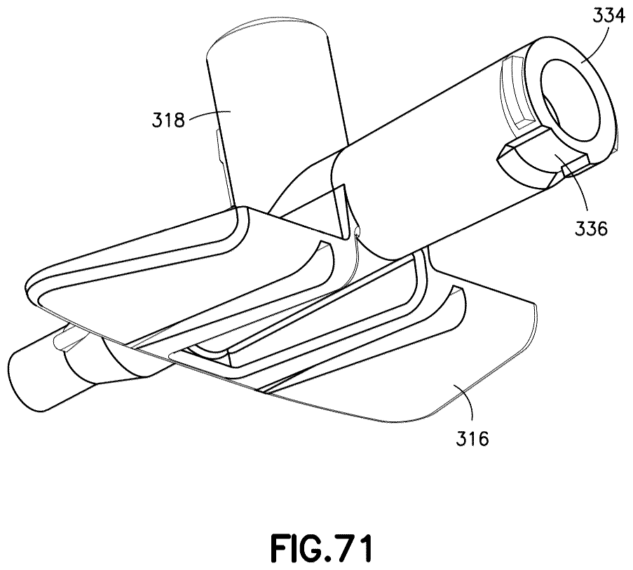

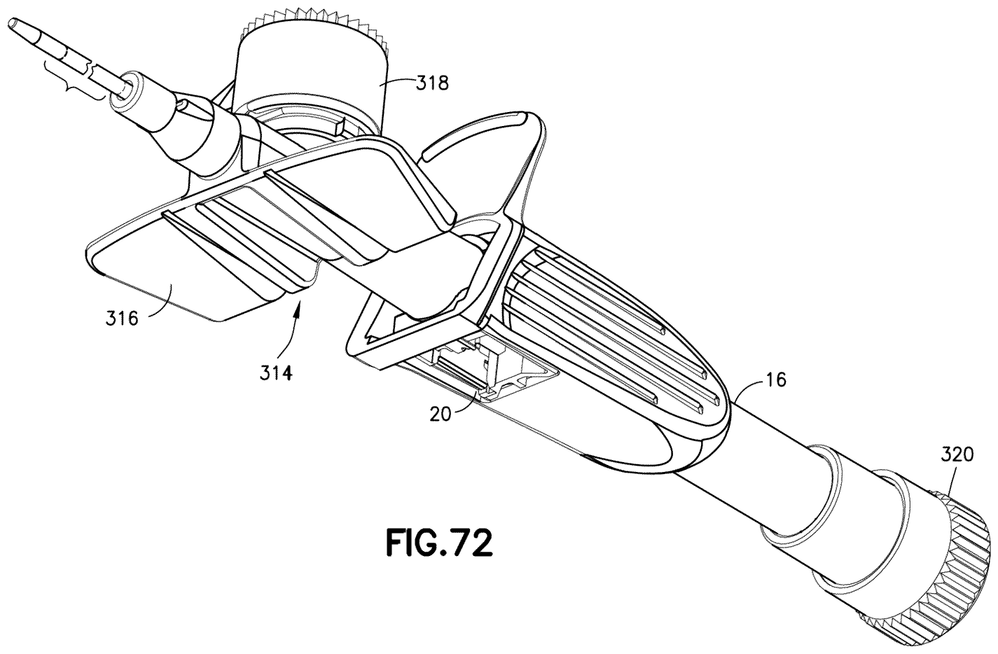

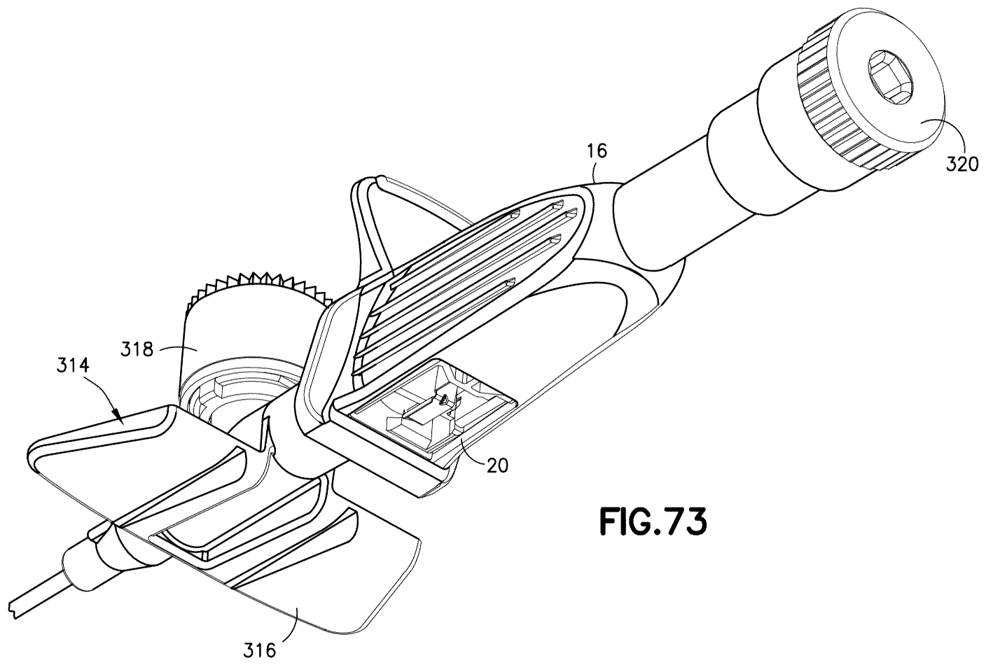

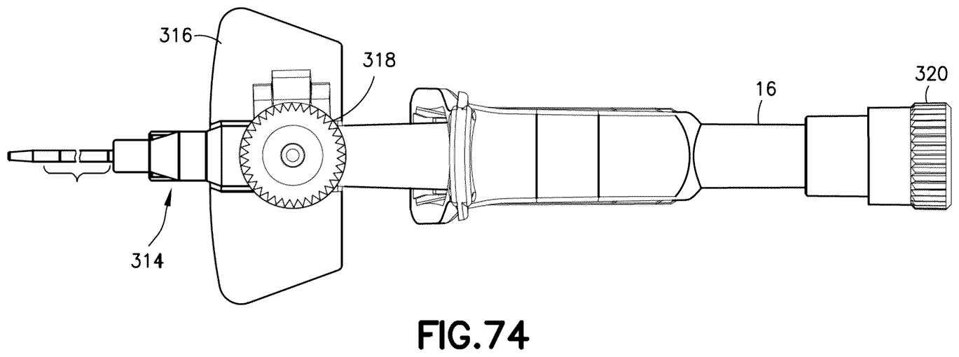

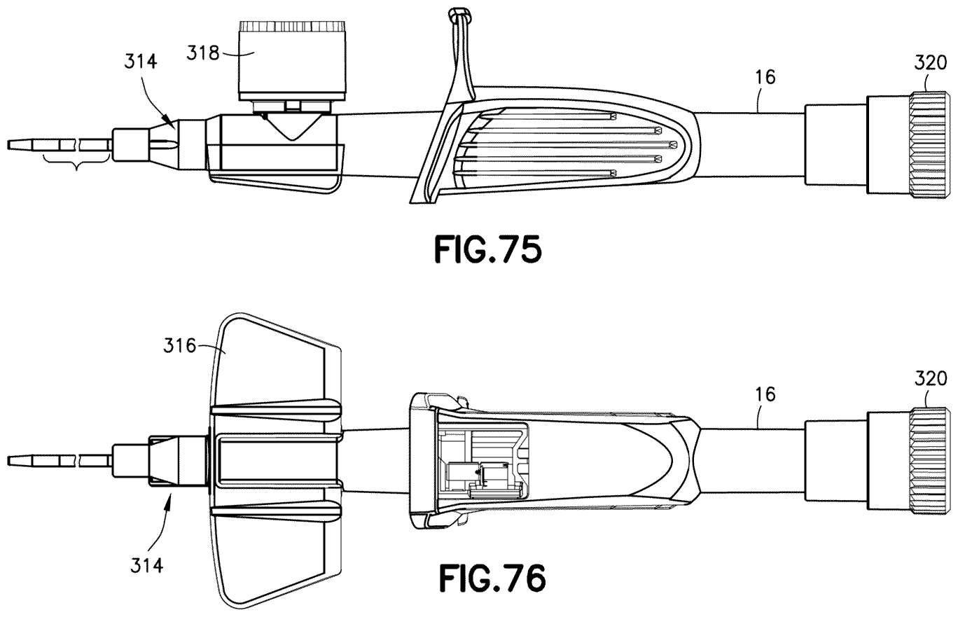

FIGS. 54-62 depict an alternative exemplary catheter hub 214 having a pair of stabilization wings 216. FIGS. 63-76 depict another alternative exemplary side port catheter hub 314 having a pair of stabilization wings 316 and a side port 318. The side port communicates with an internal tubular valve (not shown) as described in U.S. Pat. No. 4,231,367, which is incorporated herein by reference. The alternative catheter hubs 214, 314 each have a collar 234, 334 with an opening 236, 336 to receive the latch 84 of the needle shield 20.

The catheter assemblies can include a plug 320 that is initially attached to the needle hub 16. After the needle hub 16 and needle shield 20 have been removed from the catheter hub, the plug 320 can be removed from the needle hub 16 and attached to the open, proximal end of the catheter hub. Although depicted with only the side port catheter 314, the plug 320 can be used with any of the catheter hubs 14, 214, 314.

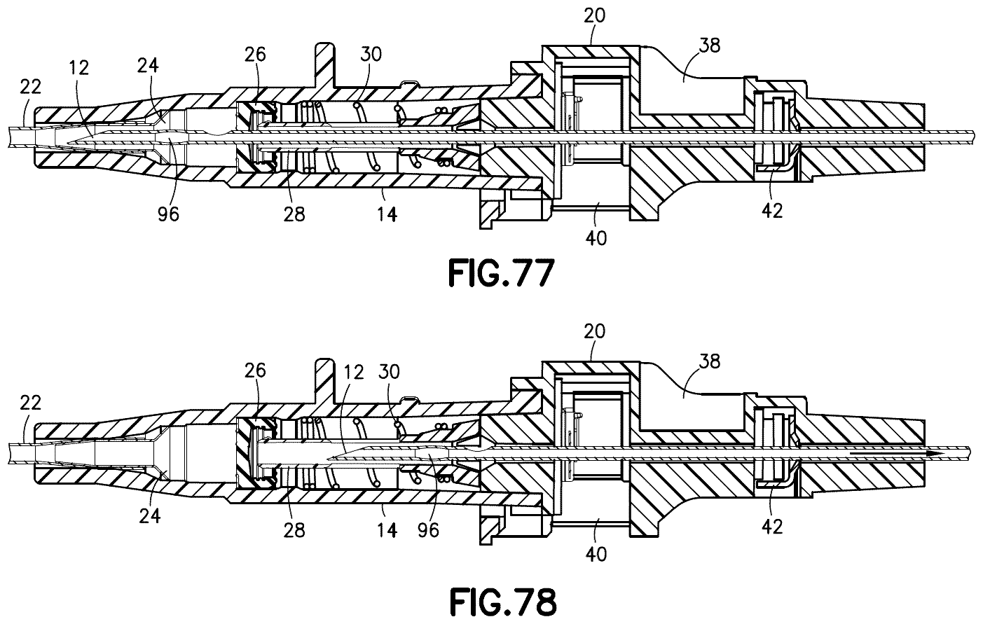

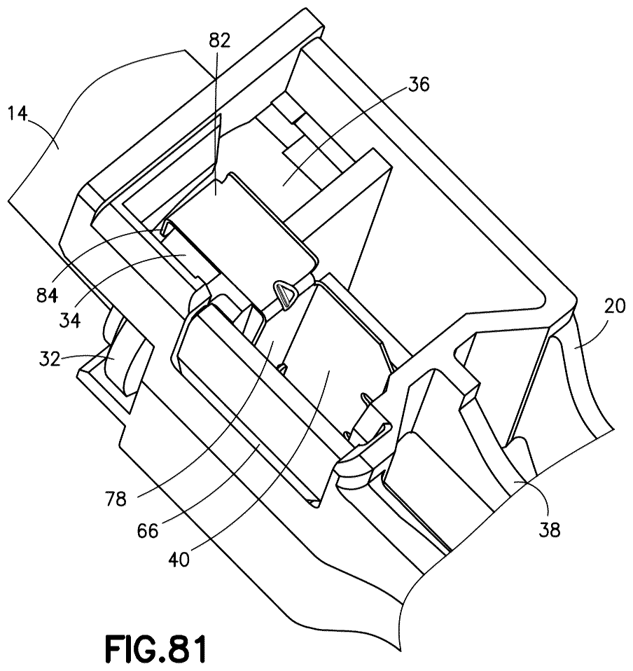

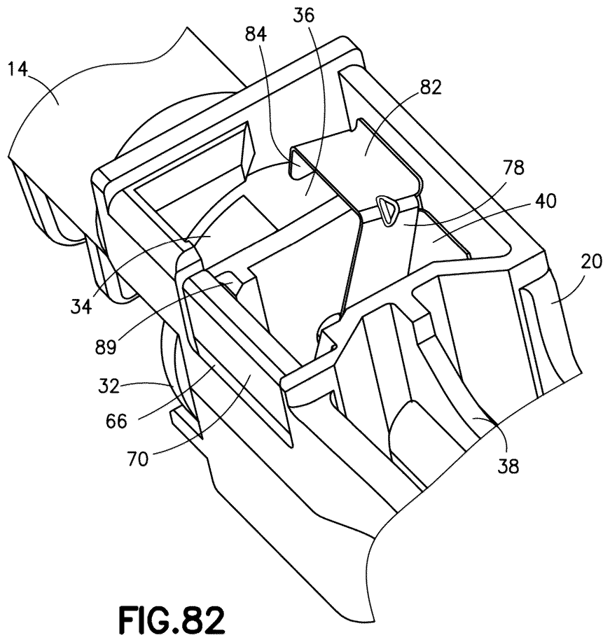

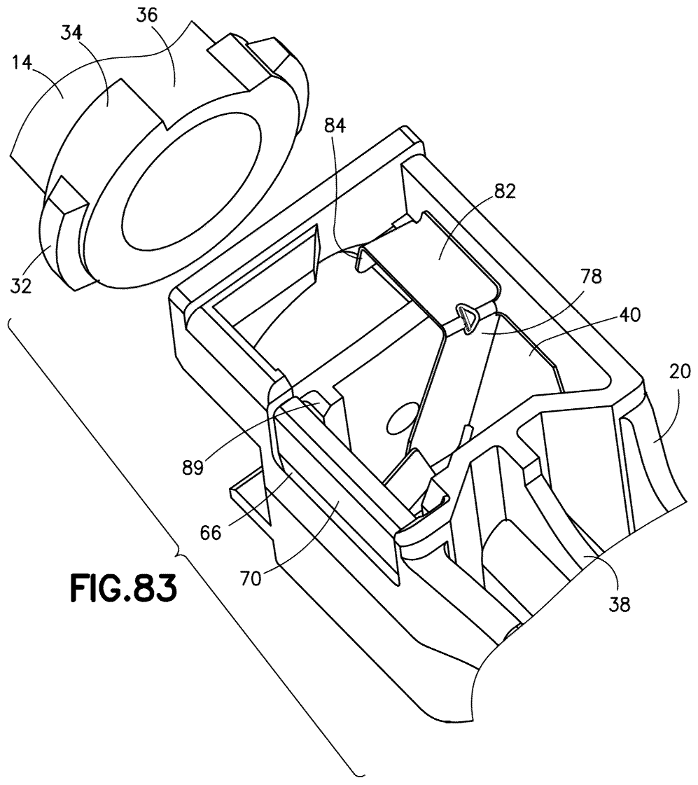

FIGS. 77-85 depict the catheter assembly 10 of FIGS. 1-12 during operation. Initially, the needle shield 20 is connected to the catheter hub 14 and the introducer needle 12 passes through the catheter hub 14 and the needle shield 20. The nose 48 of the needle shield 20 (labeled in FIG. 30) may or may not extend into the catheter hub 14 when the needle 12 is in use (first position). The needle 12 cooperates with the clip 40 by biasing the clip 40 into a locked position via pressing the first and second arms 60, 62 toward one another. In the locked position, the latch 84 engages the collar 34, preventing removal of the needle shield 20 from the catheter hub 14, as best shown in FIG. 81. At the same time, in the locked position, the latch 84 is offset from the collar opening 36. The position of the latch 84 is off-center with respect to the needle 12. The clip 40 is also in an open position, allowing the needle 12 to traverse through the clip 40.

As the needle 12 is withdrawn from the catheter hub 14 and into the needle shield 20, the tip of the needle 12 clears the clip 40, and the clip 40 is allowed to resiliently expand, causing the second arm 62 to move away from the first arm 60. As the clip 40 expands laterally, the primary and secondary flags 78, 80 block the distal opening 44 of the outer housing 38 aperture, preventing the tip of the needle 12 from exiting the distal end of the outer housing 38.

Movement of the second leg 62 moves the latch 84 laterally from engagement with the collar 34 to a position aligned with the collar opening 36, allowing the needle shield 20 to be disengaged or unlocked from the catheter hub 14. The direction in which the latch 84 moves is lateral with respect to a centerline of the needle 12. The latch 84 movement is not radial toward or away from the needle 12. Moreover, as the latch 84 is adjusted, the latch 84 moves to a centered position and then ultimately moves off-center with respect to the needle 12. The off-center positions of the latch 84 in the first and second positions of the needle 12 are symmetrically opposite each other.

In the position when the flags 78, 80 block the needle 12, the clip 40 moves to a closed position. At the same time, the needle 12 enters into a second position that is retracted from the first needle position, which prevents further use of the needle 12. The first position, as described above, is understood as, for example, all positions of the needle 12 prior to entering the second position.

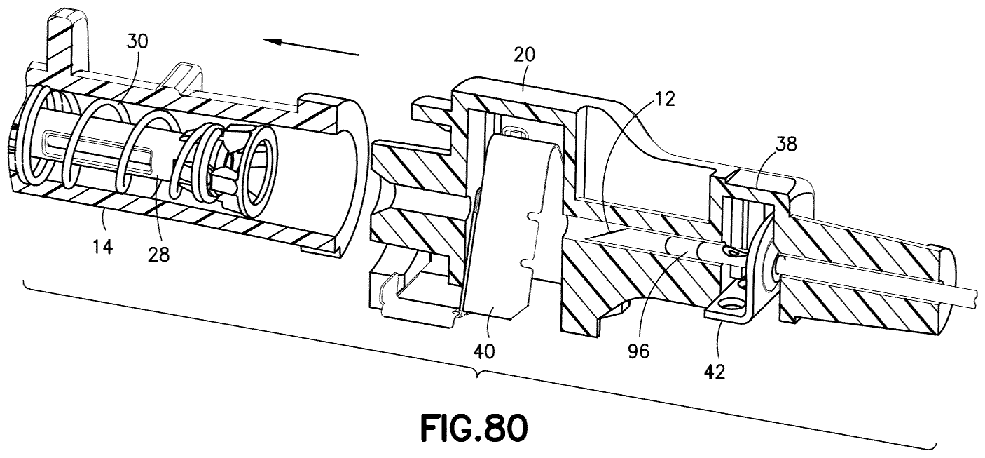

As the needle 12 is pulled further in the proximal direction, the shaft of the needle 12 slides through the needle shield 20 until the deformation 96 formed near the distal end of the needle 12 cooperates with and engages the washer 42, as shown in FIG. 80. The opening in the washer 42 is sized to allow passage of the needle shaft, but not the deformation 96. Thus, the washer 42 prevents the distal tip of the needle 12 and the deformation 96 from exiting the needle shield 20 when the needle 12 is in the second position. The combination of the washer 42 and the needle shield 20 enclose the distal tip of the needle 12 in this second position. Further proximal movement of the needle 12 results in the needle shield 20 being pulled away from the catheter hub 14.

The combination of the clip 40 and the washer 42 act as an exemplary needle tip protection mechanism. This needle tip protection mechanism encloses the distal needle tip and the deformation 96 and prevents these portions of the needle 12 from exiting the needle shield 20.

More information regarding needle tip protection mechanisms of the type used in this embodiment can be found in U.S. Pat. Nos. 6,749,588 and 7,604,616, and U.S. Patent Application Publication No. 2014/0364809, the contents of which are hereby incorporated by reference. The features described in this embodiment, including the needle protection features, can be used in combination with the features described throughout this application.

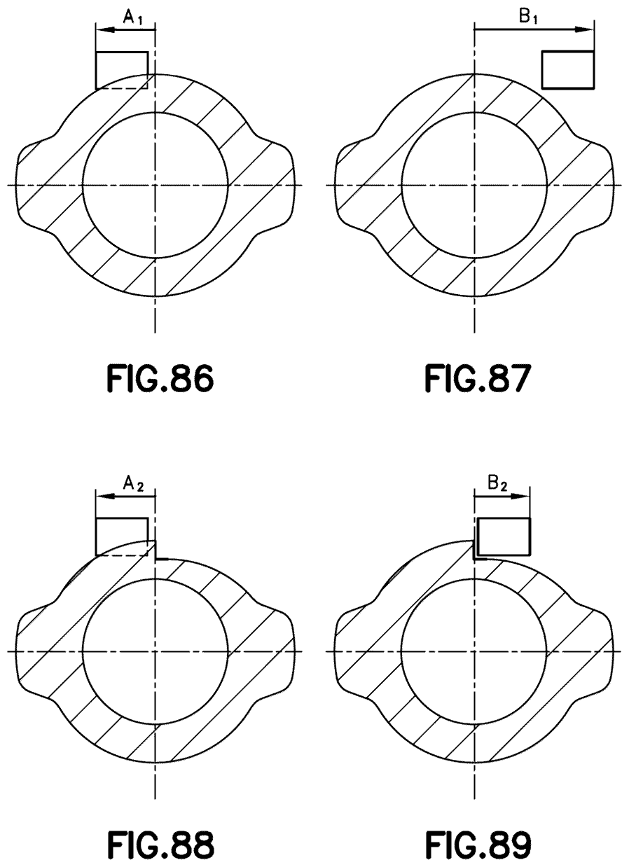

As depicted in FIGS. 86-89, the use of the clip 40 and the notched collar 34 allows for a smaller, more compact design. Without the collar opening 36, the latch 84 would have to move a distance B1 to clear the collar and allow disengagement of the needle shield 20. With the collar opening 36, the latch 84 does not have to clear the entire catheter hub 14 and only needs to move a distance B2 which is less than B1.

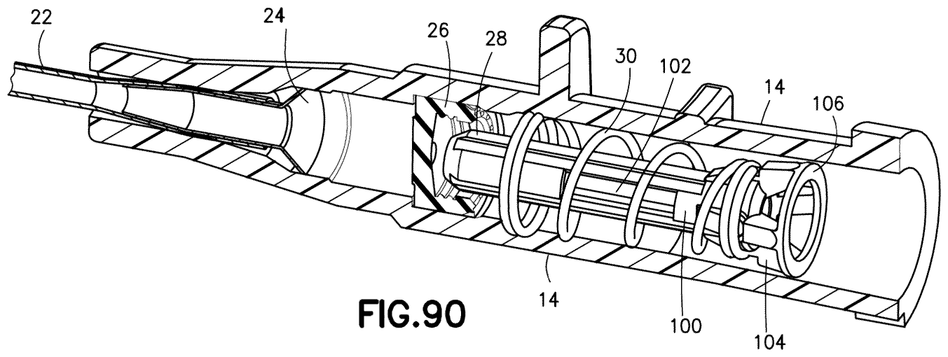

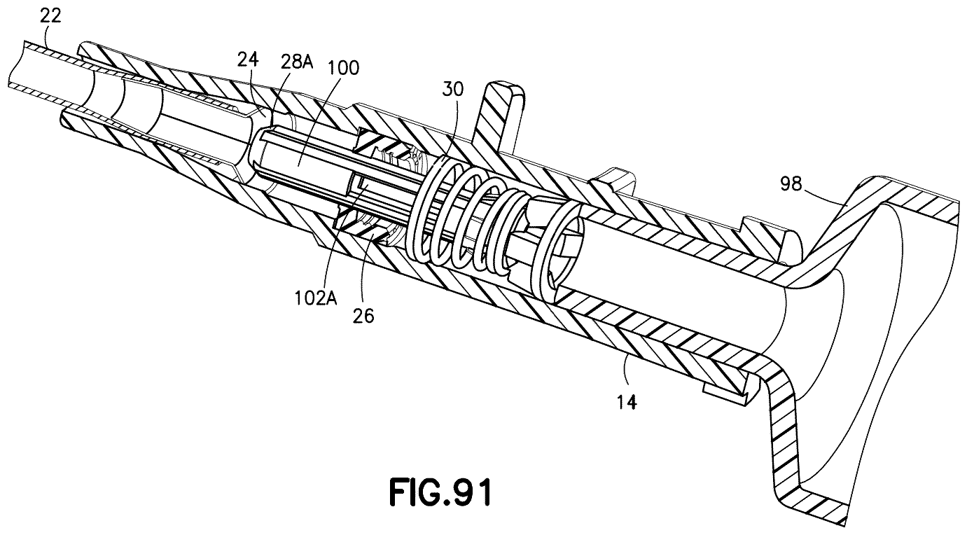

FIGS. 90 and 91 depict the use of the catheter valve actuator 28. The introducer needle 12 initially extends through the actuator 28, the septum 26, the wedge 24, and the catheter tube 22. After the introducer needle 12 and the catheter tube 22 are inserted into a patient, the needle 12 is withdrawn, closing the septum 26. As a male Luer connector 98 is inserted into the catheter hub 14, the Luer connector 98 abuts and moves the actuator 28 in the distal direction, compressing the biasing member 30. Further insertion of the Luer connector 98 moves the actuator 28 through the septum 26, opening the slits and allowing fluid to flow through the catheter hub 14.

When the Luer connector 98 is removed, the biasing member 30 moves the actuator 28 in the opposite direction, removing it from the septum 26, closing the slits, and preventing fluid from flowing therethrough. This allows the catheter to be reused while in the patient's vein, as opposed to a single-use catheter where the actuator would remain in the septum after a Luer connector 98 is removed. However, a single-use catheter can also be used with the needle shield 20 described herein.

The actuator 28 has an actuator barrel 100 surrounding an internal passage. The actuator barrel 100 is a substantially tubular member and the internal passage is substantially cylindrical. A first end of the actuator barrel 100 has a nose with a chamfered outer surface to engage the septum 26. The tubular member has one or more openings 102 to permit fluid flow through and around the actuator barrel 100. The actuator 28 includes a rear portion for engaging a male Luer connector.

In a first exemplary embodiment shown in FIG. 90, the actuator 28 includes first and second sets of openings 102 in the barrel with the first set of openings near the nose. Openings are also illustrated in the actuator 28 of FIGS. 79-80. The rear portion of the actuator 28 of FIG. 90 also includes a set of legs 104 extending from the barrel and connected to a ring 106. The features described in this embodiment can be used in combination with the features described throughout this application.

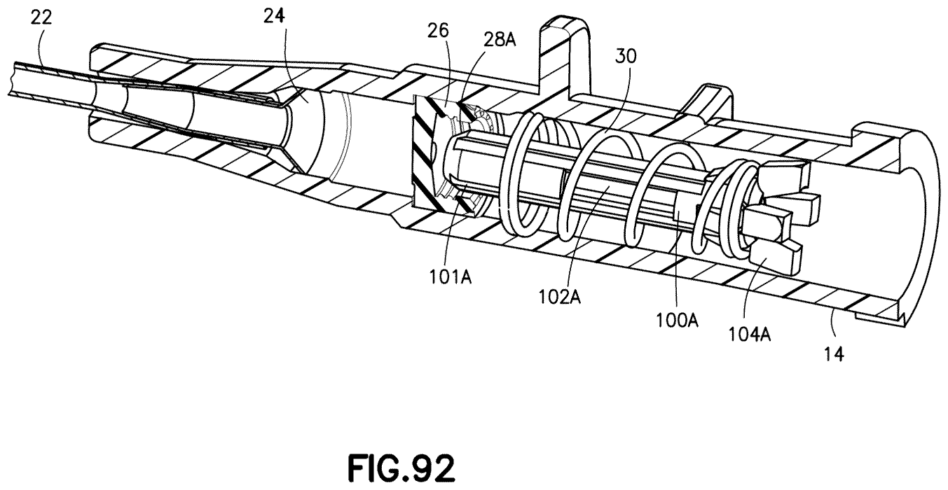

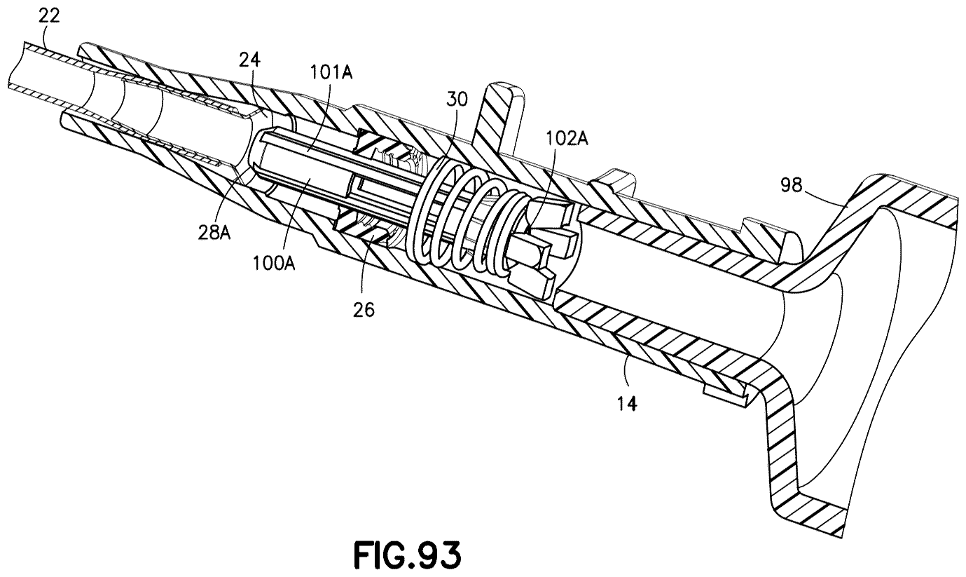

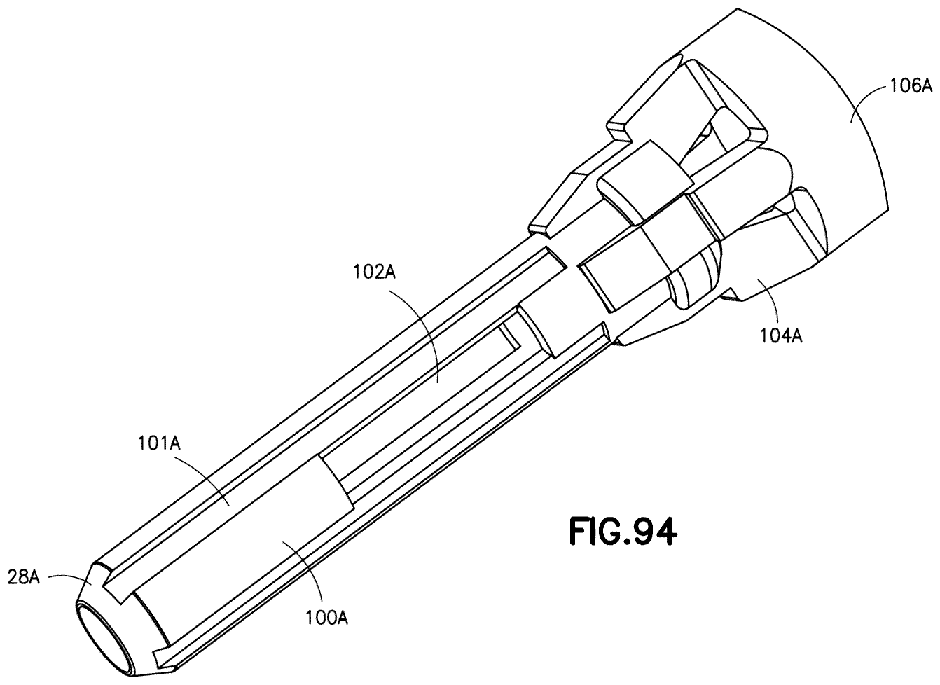

In a second exemplary embodiment shown in FIGS. 92-94, the actuator 28A includes a set of grooves 101A and a set of openings 102A. The grooves 101A extend from the nose toward the back of the actuator barrel 100A. The openings 102A are positioned towards the rear of the barrel 100A. When the actuator 28A extends through the septum 26, the grooves 101A channel fluid to the openings 102A which remain on the proximal side of the septum 26. The grooves 101A may be positioned on the side of the openings 102A or directly in line with the openings 102A. The rear portion of the actuator includes a set of legs 104A extending from the barrel. As illustrated in FIG. 94, a ring 106A may be connected to the legs 104A to engage a Luer connector 98 or the Luer connector 98 may directly engage the legs 104A as illustrated in FIGS. 92 and 93. The features described in this embodiment can be used in combination with the features described throughout this application.

In an exemplary embodiment, the biasing member 30 is a spring, for example a helical compression spring with a distal end and a proximal end. The spring may be made from metal, plastic, an elastomer, or another suitable resilient material. The distal end of the spring forms an interference fit with the inner surface of the catheter hub 14. The interference fit may be sufficient to retain the spring, even during loading. The proximal end of the spring connects to the actuator 28. The features described in this embodiment can be used in combination with the features described throughout this application.

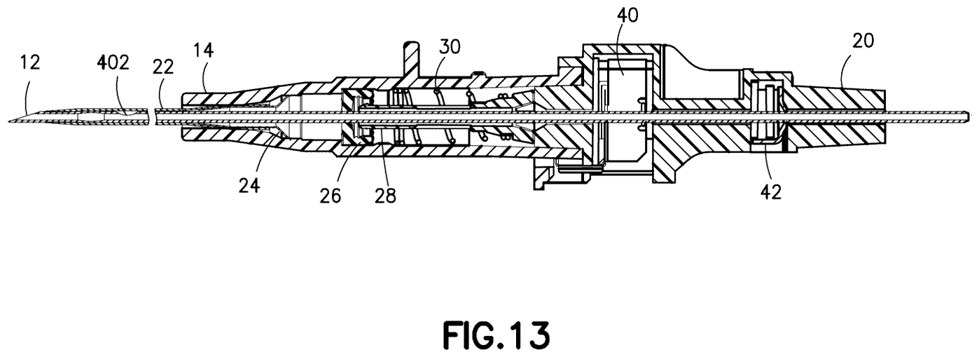

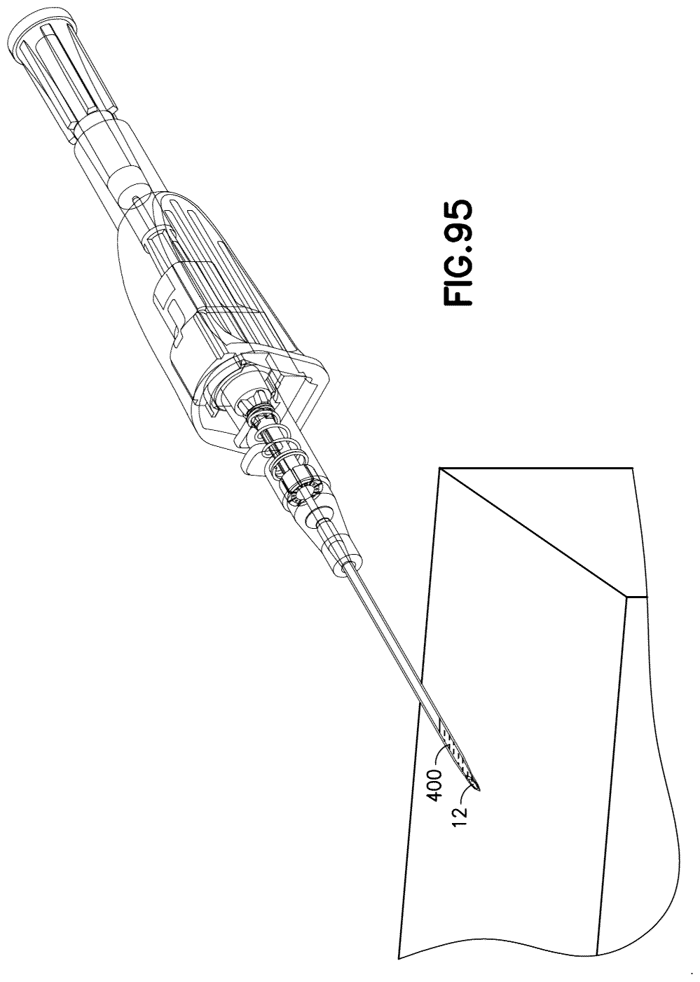

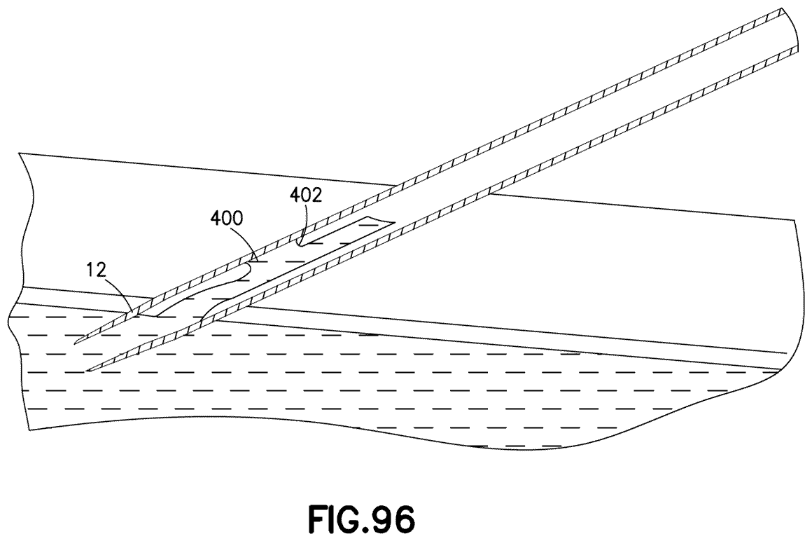

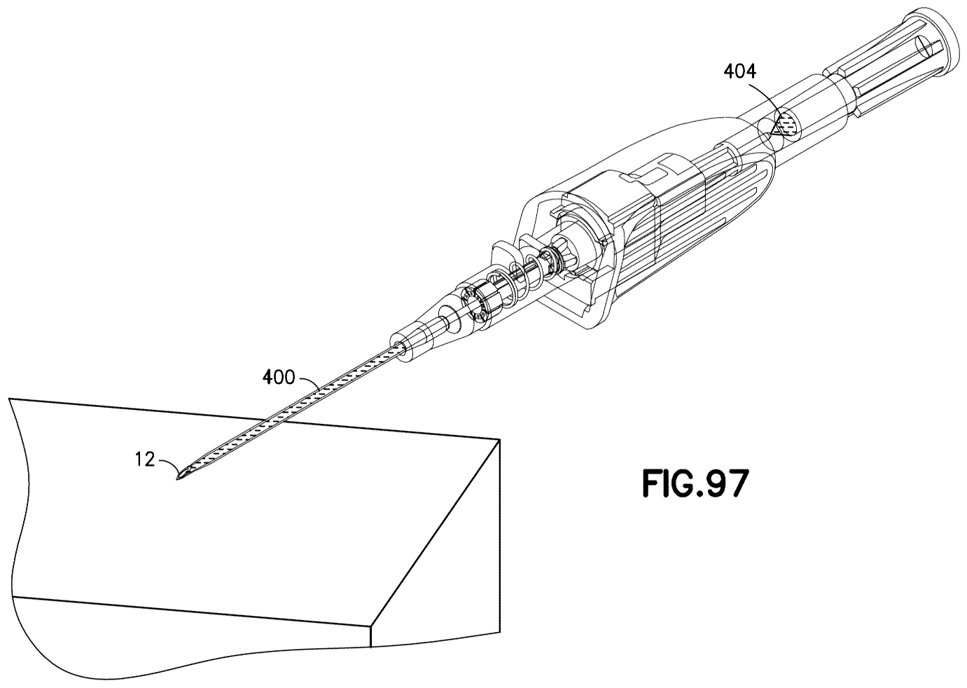

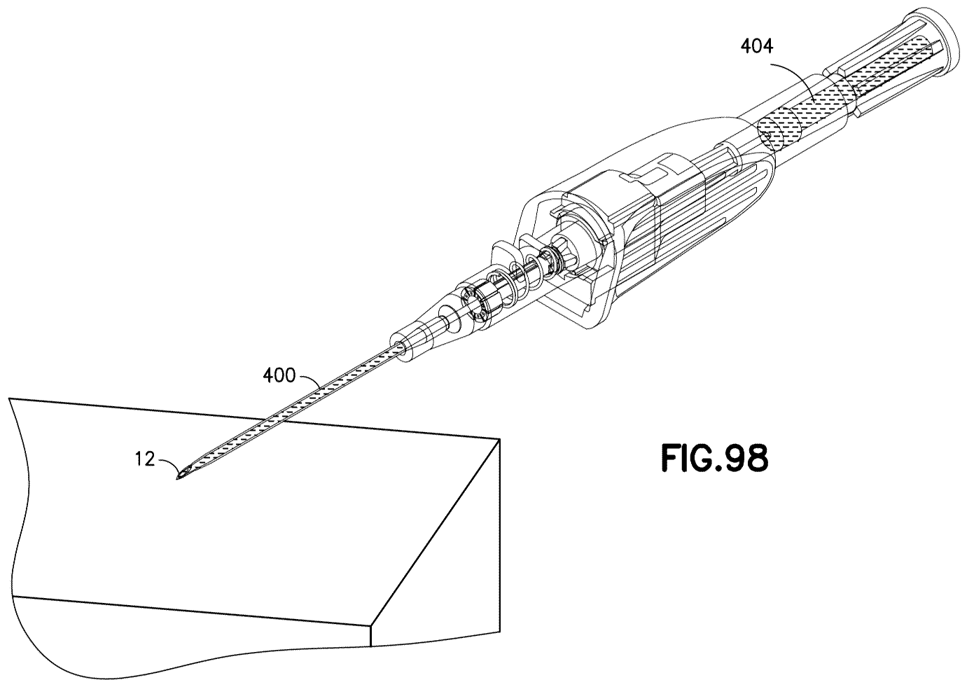

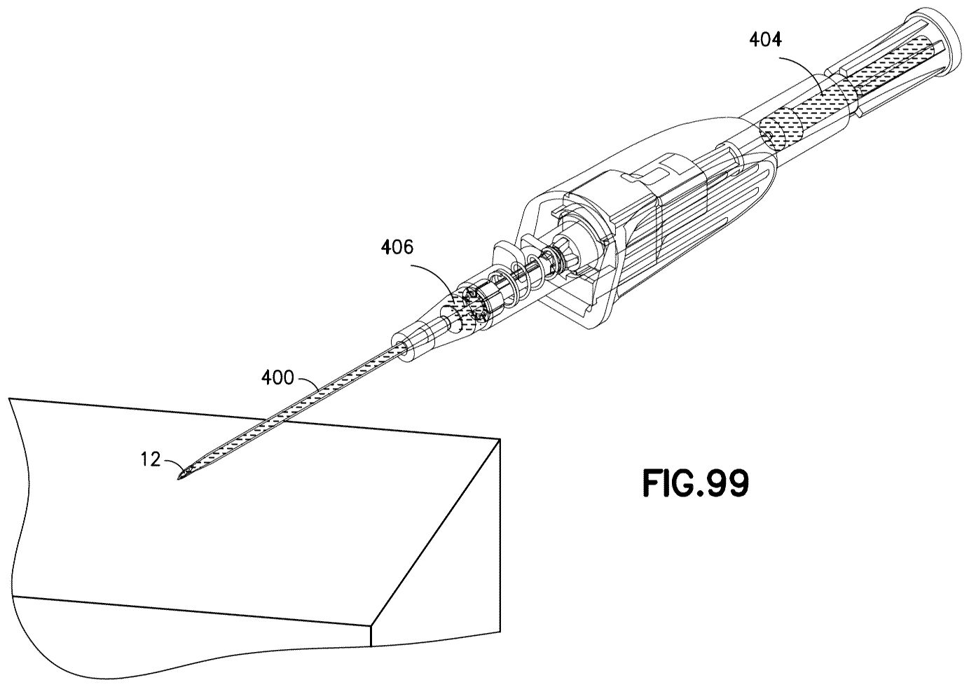

FIGS. 95-99 depict various exemplary blood flashback features of the catheter assembly. Flashback is the visibility of blood that confirms the entry of the needle tip into the vein. Primary flashback 400 is seen through the catheter tubing as blood travels into the open distal end of the hollow needle 12, out a notch or opening 402 (also visible in FIG. 13) in the needle 12 near the needle tip, and up through the internal annular space between the needle 12 and the inside of the catheter tubing 22. The secondary flashback 404 is seen in the needle hub/grip 16 when it comes out of the back of the needle 12 and enters a flash chamber in the needle hub/grip. Air is vented by the plug in the back of the needle hub/grip 16 by a porous membrane or micro grooves. Tertiary flashback 406 is in the catheter hub 14 when the blood from the primary flashback 400 flows into it and stops at the blood control septum 26. Air is vented by micro grooves in the periphery of the blood control septum 26. The features described in this embodiment can be used in combination with the features described throughout this application.

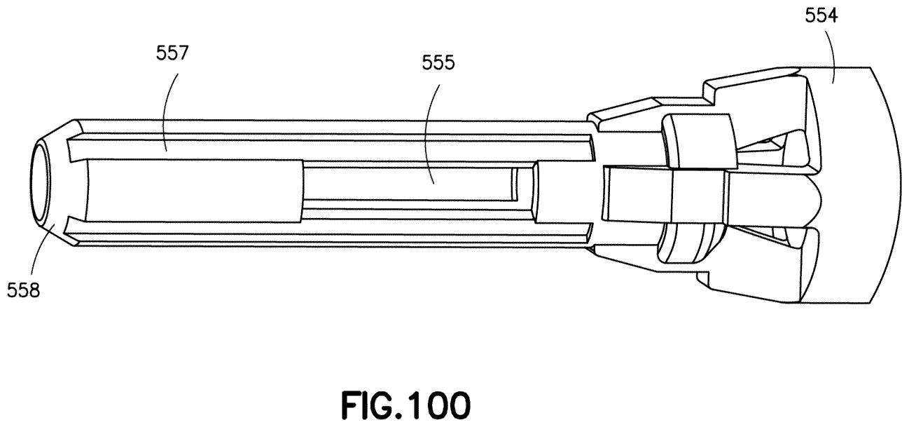

FIG. 100 illustrates the actuator of FIG. 94 in further detail. The actuator 554 can be used in the catheter assemblies illustrated in FIG. 90-93. The actuator 554 includes a nose 558 that reduces friction when the actuator 554 penetrates into a septum 538 of a catheter hub assembly. The actuator 554 further includes openings 555 that extend through the actuator 554 in a direction perpendicular to a centerline of the actuator 554. For example, the actuator 554 can include two rectangular shaped openings 555, although more or less are contemplated.

The actuator 554 also includes a plurality of grooves 557 that extend axially along the distal portion of an outer surface of the actuator 554 in a plane parallel to the centerline of the actuator 554. For example, four grooves 557, substantially radially equidistant from each other, can be present along an external surface of the distal portion of the actuator 554, although more or less grooves 557 are contemplated. The grooves 557 can be of varying depths into the actuator 554. The grooves 557 are different from the openings 555 because the grooves 557 do not extend through the actuator 554.

The openings 555 and the grooves 557 advantageously provide increased area for the fluid to move inside the catheter hub assembly. The increased area advantageously allows for fluid flushing and to prevent coagulation of fluid in the proximal and distal ends of the septum. Additionally, the openings 555 and the plurality of grooves 557 advantageously minimize the stagnation of fluid and allow for greater mixing. The grooves 57 further prevent the septum from sealing on an outside surface of the actuator during operation. By not forming a sealing interface, the fluid is permitted to leak through the septum via the grooves 57 and provide additional flushing.

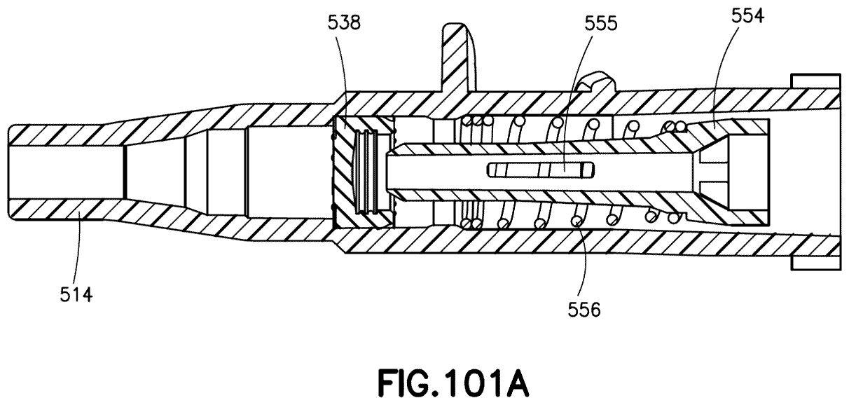

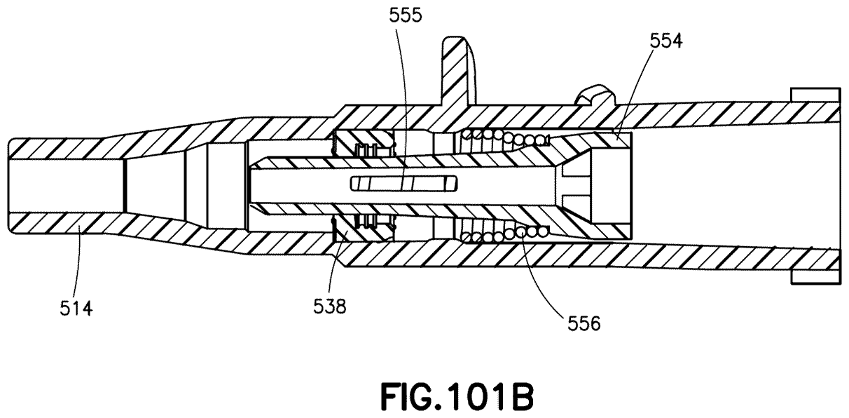

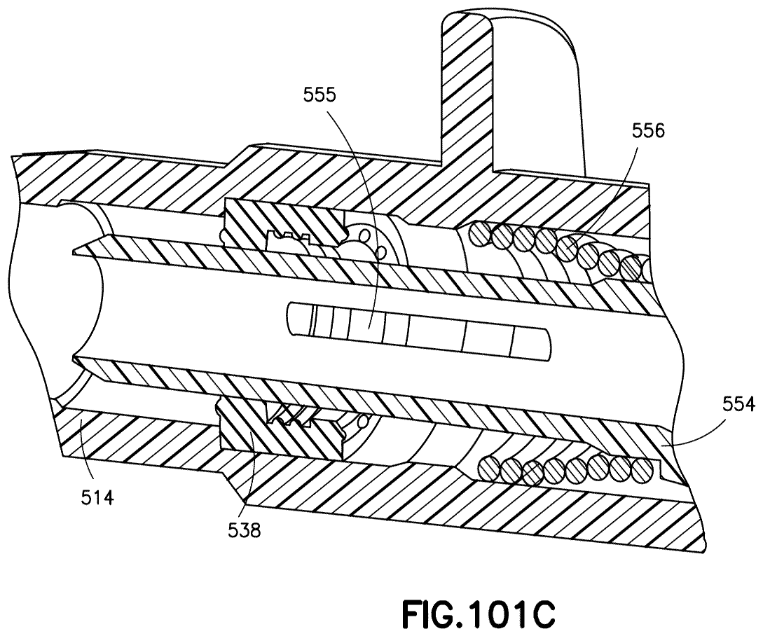

FIG. 101A illustrates the actuator 554 of FIG. 100 in the catheter hub assembly. Similar to the embodiments described above, the catheter hub assembly further includes a catheter hub 514, a septum 538 and a biasing member 556. As illustrated, the openings 555 and the grooves 557 of the actuator 554 provide more area for fluid flow inside the catheter hub 514, thus achieving the advantages described above.

FIGS. 101B and 101C illustrate the catheter hub assembly when the biasing member 556 is compressed and the actuator 554 penetrates the septum 538. The catheter hub assembly may be configured such that the openings 555 and/or the grooves 557 of the actuator 554 optionally penetrates the septum 538. In this embodiment, the openings 555 in the actuator 554 do not penetrate the septum 538. However, the grooves 557 in the actuator 554 penetrate the septum 538. This configuration allows for increased fluid flow from the proximal end to the distal end of the septum 38 through the grooves 557, in addition to the advantages described above. After operation of the catheter assembly is complete, the actuator 554 is retracted from the septum 538 via the force exerted by the biasing member 556. The catheter assembly is configured for multiple uses upon depression of the actuator 554. The features described in this embodiment, including the actuator, can be used in combination with the features described throughout this application.

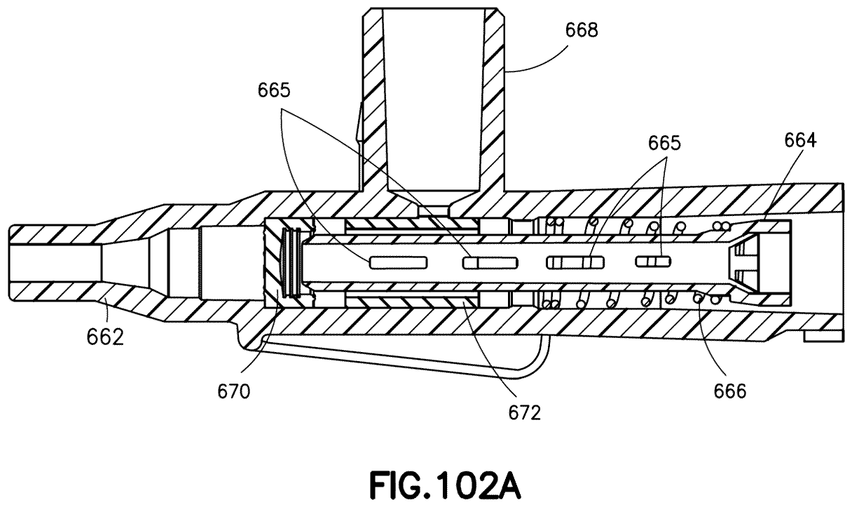

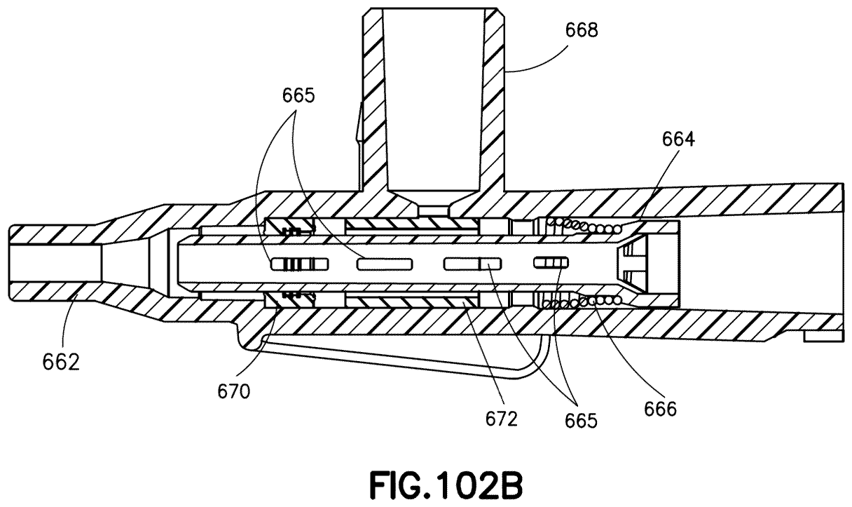

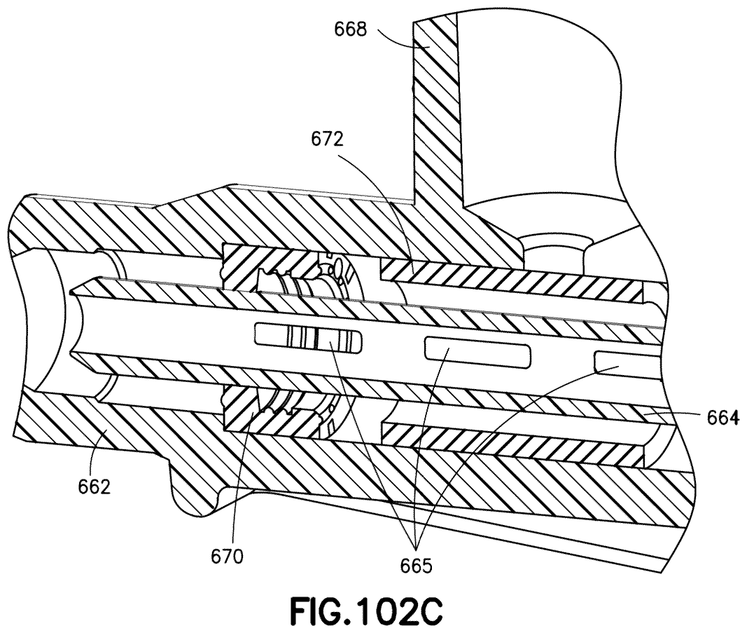

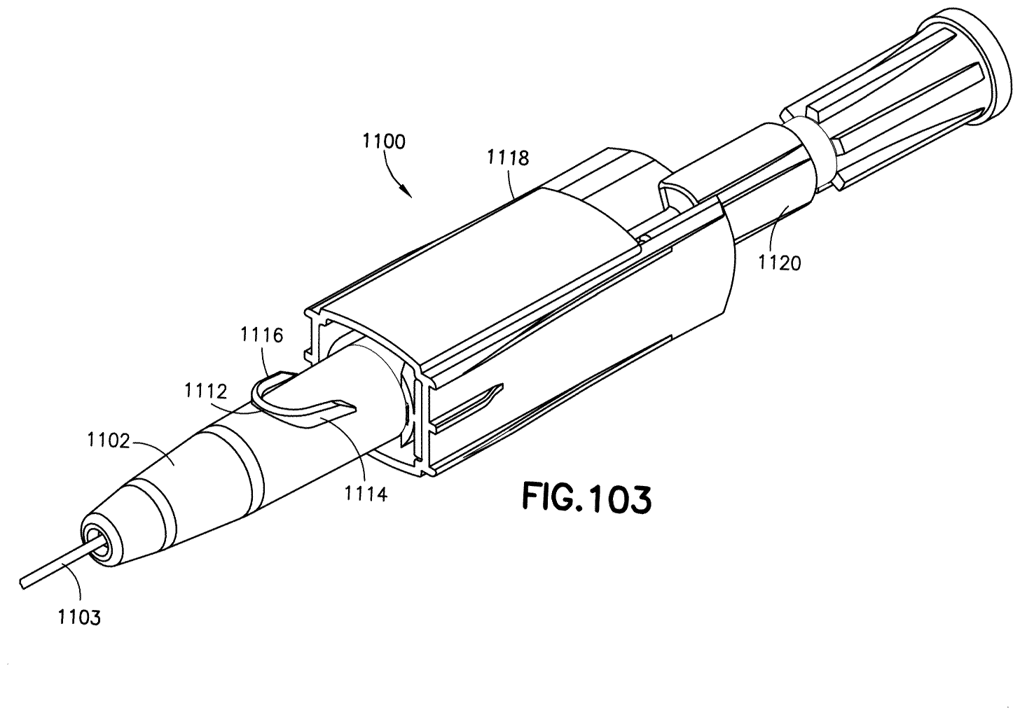

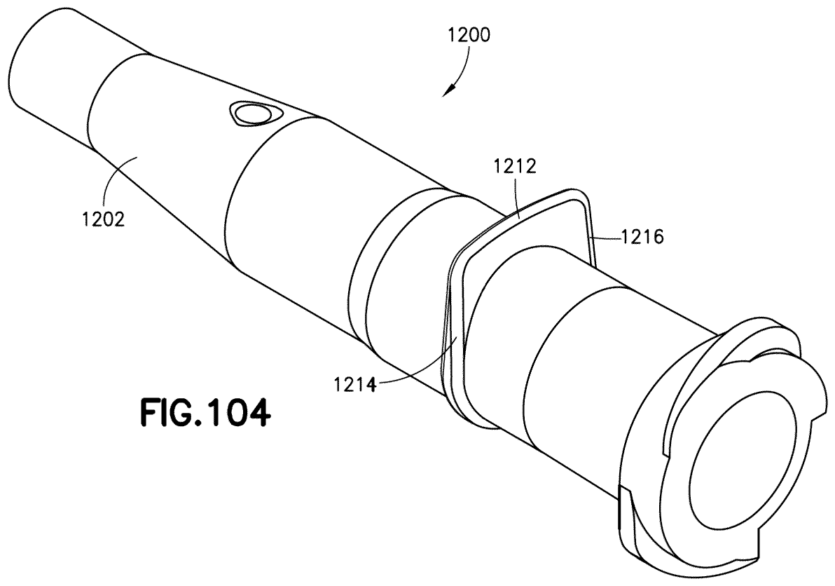

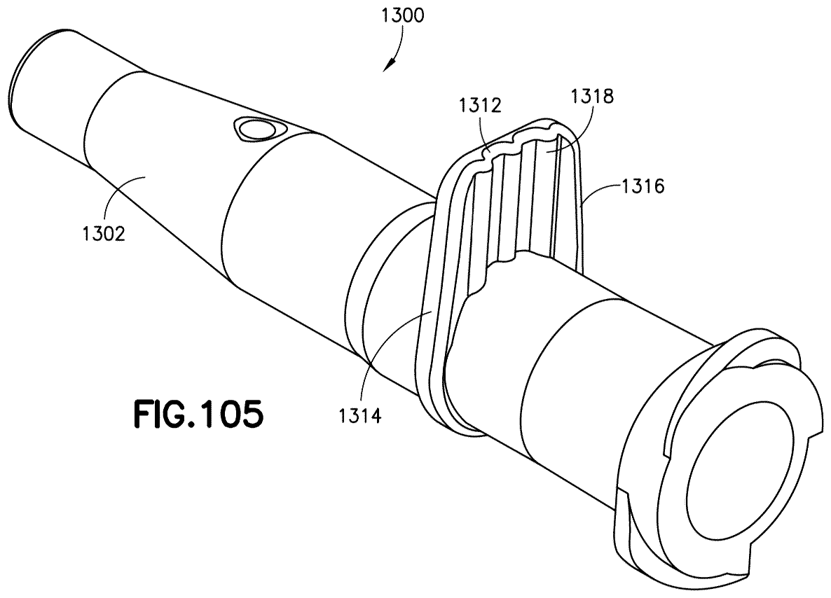

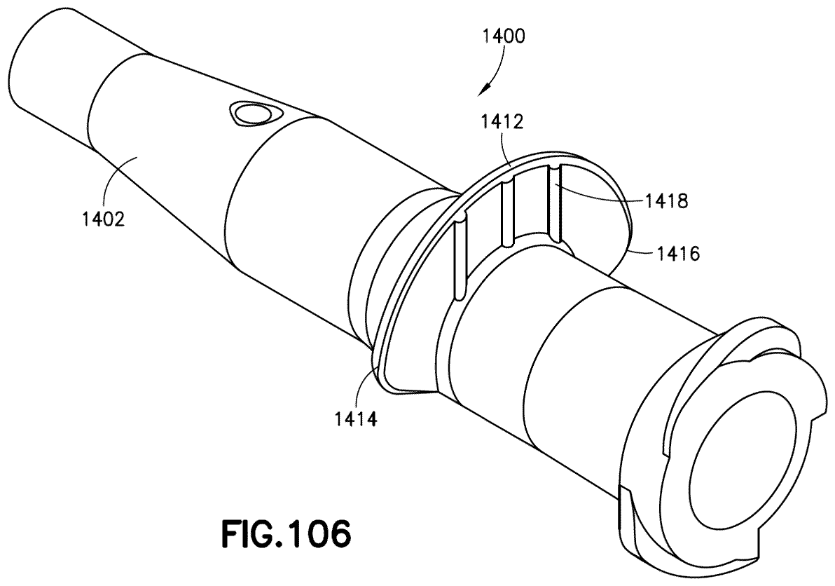

FIG. 102A illustrates another embodiment of an actuator 664 in a catheter hub assembly. The catheter hub assembly includes a catheter hub 662 having a side port 668. The side port 668 provides secondary access to the fluid flow in the catheter hub 662. The intersection of the main bore of the catheter hub 662 and the side port 668 includes a sleeve 672. The sleeve 672 provides selective fluid communication between the side port 668 and the catheter hub 662. Specifically, when sufficient fluid pressure is applied through the side port 168, the sleeve 672 compresses. The compression of the sleeve 672 allows for fluid to enter the catheter hub 662. The catheter hub assembly further includes a septum 670 and a biasing member 666 that provides tension to the actuator 664.

The actuator 664 includes a plurality of openings 665 that extend through the actuator 664 in a similar manner as described above. The actuator 664 includes two rows of four openings 665 having different sizes and similar spacing, although various quantities, sizes and spacing of the openings 665 are contemplated. As illustrated, the openings 665 provide more area for fluid flow inside the catheter hub 662, thus achieving similar advantages described above with respect to FIGS. 100-101C.

FIGS. 102B and 102C illustrate the catheter hub assembly when the actuator 664 penetrates the septum 670 and compresses the biasing member 666. The catheter hub assembly is configured such that the openings 665 of the actuator 664 optionally penetrate the septum 670. In this embodiment, the openings 665 in the actuator 664 do not penetrate the septum 670. This configuration allows for increased fluid flow between the side port 668 and the catheter hub 662 at the proximal end of the septum 670, in addition to the advantages described above. If the openings 665 in the actuator 664 penetrate the septum 670, increased mixing of fluid would also take place at a distal end of the septum 670.

When operation of the catheter assembly is complete, the actuator 664 is retracted from the septum 670 via the force exerted by the biasing member 666. The catheter assembly is configured for multiple uses upon depression of the actuator 664. The features described in this embodiment, such as the actuator, can be used in combination with the features described throughout this application.

In another exemplary embodiment, the collar of the catheter hub as described above can be replaced by any other structure that defines a notch. For example, the collar may be a groove or a recess in the catheter hub. Accordingly, the groove in the catheter hub can be used to engage and disengage a clip in a similar manner as described above. The features described in this embodiment can be used in combination with the features described throughout this application.

Referring in more specific detail to FIG. 103 of the drawings, there is illustrated a medical device such as a safety IV catheter assembly 1100 incorporating a catheter hub (hub) 1102 and a flexible tube or cannula 1103. The catheter hub 1102 is releasably engaged to a needle tip shield 1118 (housing). The catheter tube 1103 is directly or indirectly connected to the hub 1102 or housing 1118. The material of the catheter tube 1103 may consist of, for example, polyurethane (PU), FEP or PTFE (Teflon™). For purposes of illustration, the catheter hub 1102 is shown attached to an introducer needle hub 1120 prior to insertion. The catheter hub 1102 includes a push tab 1112 and anti-rotation features in the form of extension members 1114 and 1116. A user can engage the push tab 1112 to advance the catheter hub 1102 forward thereby advancing the catheter. As illustrated in FIG. 1, the push tab 1112 extends radially from an upper surface of the catheter hub 1102. The first extension 1114 extends from a first side of the push tab 1112 around the side of the upper surface of the catheter hub 1102 and the second extension 1116 extends from a second side of the push tab 1112 around the other side of the upper surface of the catheter hub 1102. The first extension 1114 and the second extension 1116 act as anti-rotation members that counteract rotation of the catheter hub 1102. The push tab and first and second extensions together form a substantially C-shape when viewed from above.

The push tab and first and second extensions are shaped and configured to cradle a user's finger as the push tab 1112 is advanced forward. The first extension 1114 and second extension 1116 resist angular rotation of the catheter hub 1102 relative to the user's finger and enhance stability during insertion.

In an alternate embodiment (not shown), the push tab 1112 and extension members 1114, 1116 are similarly disposed on a top distal surface 1122 of the needle tip shield 1118, instead of on the catheter hub 1102. The introducer needle hub 1120 includes an opening at a top distal surface to allow the push tab 1112 and extension members 1114, 1116 of the needle tip shield 1118 to extend upwardly and be accessible to the user. The catheter tube 1103 is directly or indirectly connected to the hub 1102 or housing 1118. Accordingly, the user can engage the push tab 1112 on the needle tip shield 1118 to advance the catheter hub 1102 and catheter forward. After the catheter is inserted, the introducer needle hub 1120 is used to withdrawn the introducer needle of the catheter assembly 1100 from the catheter tube 1103 and the catheter hub 1102. Subsequently, a distal end of the introducer needle is retracted and enclosed in the needle tip shield 1118. The push tab 1112 and extension members 1114, 1116 of the needle tip shield 1118 also aid the user to withdraw the introducer needle of the catheter assembly 1100.

For this and other subsequently-described embodiments, all reference characters designating corresponding parts of the embodiments will be the same as in the embodiment of FIG. 103, except that they will be in a different series, for example, in the 1200 series, or the 1300 series. The differences of the second and third embodiments with respect to the first embodiment will now be described.

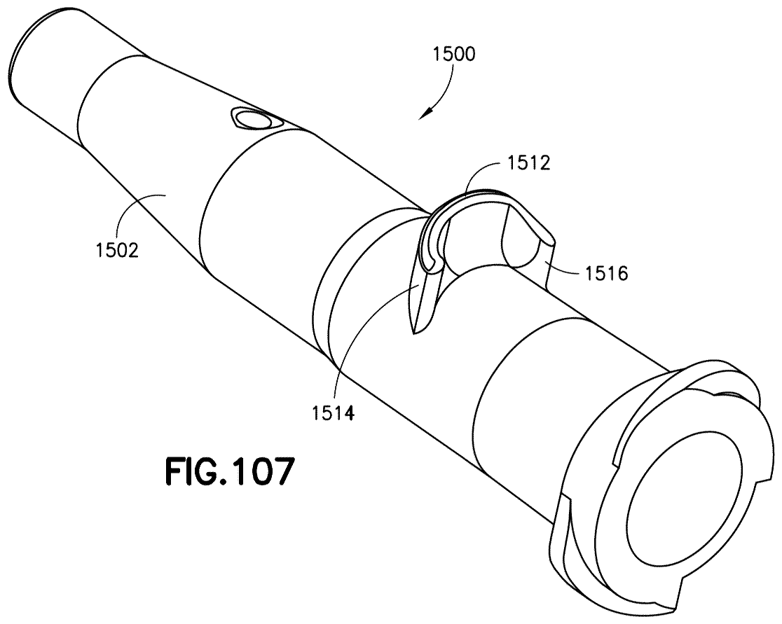

FIG. 104 depicts a push tab 1212 formed on an upper surface of a catheter hub 1202 for a catheter hub 1200. As illustrated in FIG. 104, the push tab 1212 is a wall-like formation extending radially from an upper surface of the catheter hub 1202. The push tab 1212 includes a raised and sculpted configuration where a distal side of the wall-like main portion 1212 is concave so as to conform to the curvature of the user's finger and allow the user to control rotation. A first extension 1214 extends from a first side of the wall-like main portion 1212 around an outer surface of the catheter hub 1202, and a second extension 1216 extends from a second side of the main portion 1212 around the outer surface of the catheter hub 1202. The first extension 1214 and the second extension 1216 act as anti-rotation members that counteract rotation of the catheter hub 1202. The sculpted configuration of the push tab 1212 provides a tactile feel for the user with regard to placement of the user's finger.

FIG. 105 provides a sculpted push tab 1312 on an upper surface of a catheter hub 1302. The push tab 1312 includes ribs 1318 disposed on a wall-like main portion of the tab 1312. Ribs 1318 enhance the tactile feel with regard to placement of the user's finger and assist in maintaining the user's finger on the tab 1310.

A push tab 1412 is formed on an upper surface of the catheter hub 1402 illustrated in FIG. 106 for use with a catheter. The push tab 1412 includes a wall-like main portion extending radially from an upper surface of the catheter hub 1402. The push tab 1412 also includes a first extension 1414 and a second extension 1416. The first extension 1414 and second extension 1416 both extend radially from side surfaces of the catheter hub. Together with the main portion 1412, they provide a larger circumference for the finger-engaging surface than the embodiments of FIGS. 104 and 105. At least one rib 1418 is formed on the push tab 1412 to facilitate engagement with a user's finger and prevent rotation. The first and second extension 1414 and 1416 limit rotation of the catheter hub 1402 such that as the catheter rotates either clockwise or counterclockwise, either the first extension 1414 or the second extension 1416 will contact the skin of the patient and prevent further rotation, while the push tab 1412 is still in contact with the clinician's finger, allowing advancement.

FIG. 107 illustrates a catheter hub 1502 with a more deeply sculpted push tab 1512 formed on an upper surface of the catheter hub 1502. The deep-sculpted push tab 1512 includes a wall-like main portion extending radially from an upper surface of the catheter hub 1502. A first extension 1514 and a second extension 1516 extend from the wall-like main portion of the push tab 1512. The first extension 1514 and second extension 1516 both extend proximally on the catheter hub 1502 and curve toward the main portion of the push tab 1512 to cradle a user's finger by engaging the sides of the clinician's fingers and allow the user to control lateral motion and rotation. The first extension 1514 and the second extension 1516 act as anti-rotation members that counteract rotation of the catheter hub 1502.

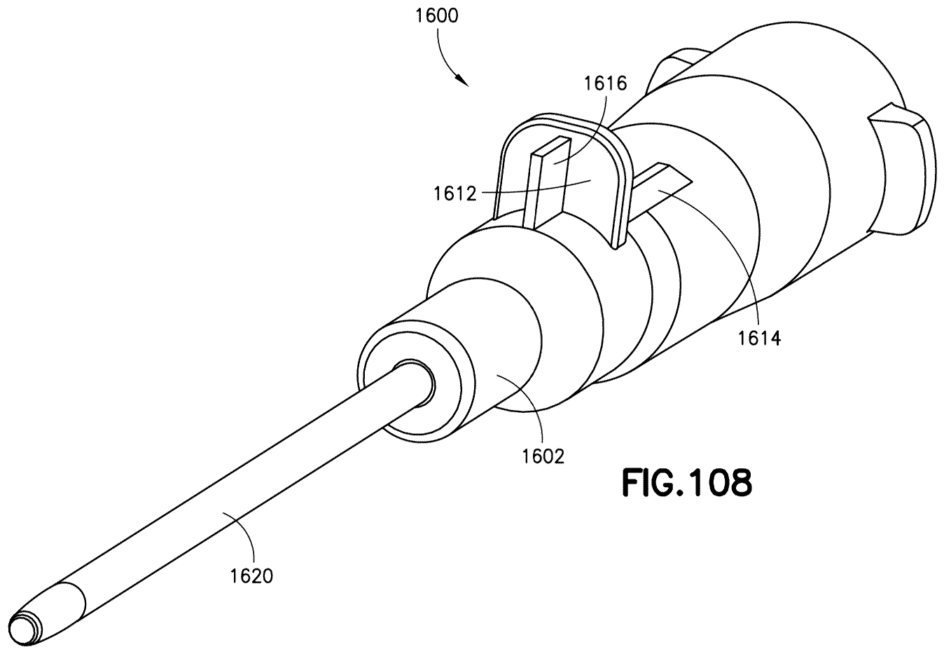

Regarding FIG. 108, a catheter hub 1602 incorporating a catheter 1620 is illustrated. A push tab 1612 is formed on an upper surface of the catheter hub 1602. As illustrated in FIG. 108, the push tab 1612 is configured as a wall-like main portion extending radially from an upper surface of the catheter hub 1602. A first cradling tab 1614 extends from a first side of the push tab 1612 perpendicular to a plane of the push tab 1612. A second cradling tab 1616 extends from a second side of the main portion 1612 perpendicular to a plane of the push tab 1612. The push tab 1612, first extension 1614 and second extension 1616 thereby form a cradle shape to resist catheter hub rotation where the first extension 1214 and the second extension 1216 act as anti-rotation members that counteract rotation of the catheter hub 1202.

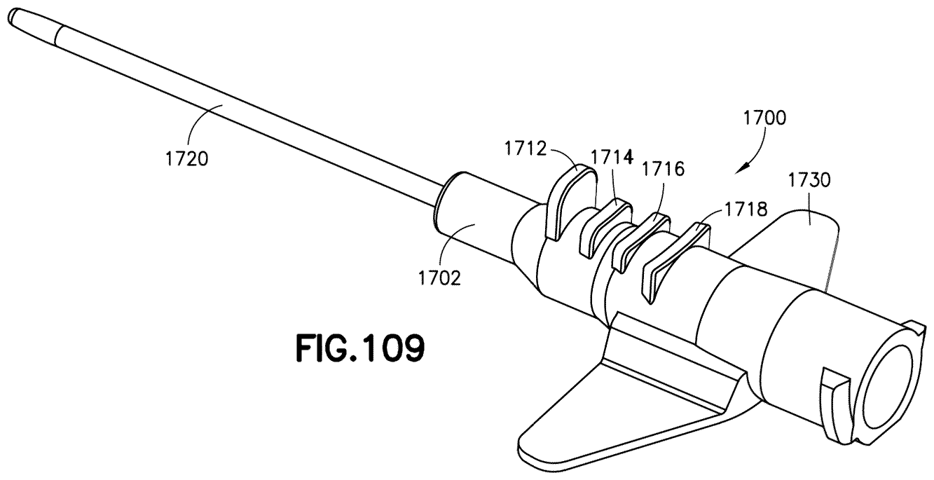

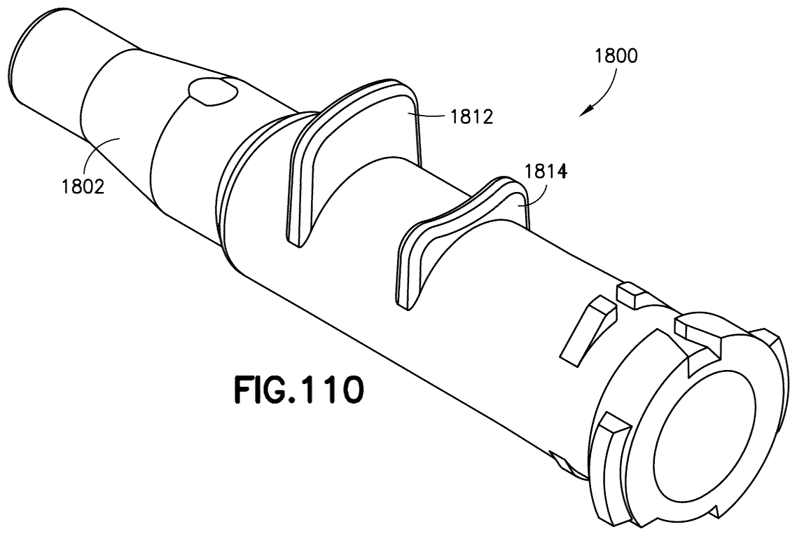

FIG. 109 illustrates a wing catheter hub 1702 incorporating a flexible IV catheter 1720 and wings 1730. A push tab 1712 is formed on an upper surface of the catheter hub 1702. As illustrated in FIG. 109, the push tab 1712 includes a tall wall-like main portion extending radially from an upper surface of the catheter hub 1702. Anti-rotation push tab 1712 provides a cradling effect for the user's finger to aid insertion stability.

A first rib 1714 parallel to the plane of the push tab 1712, but shorter in height, extends from an upper surface of the catheter hub 1702 and is spaced proximally from to the push tab 1712. A second rib 1716 and a third rib 1718, also shorter in height than the first rib 1714, may also extend from an upper surface of the catheter hub 1702 parallel to the plane of the push tab 1712. The ribs 1714, 1716, and 1718 form a cradle shape to resist catheter hub rotation. Ribs 1714, 1716 and 1718 also strengthen the catheter hub to prevent shrinkage which could cause leakage for any internal components of the catheter hub requiring a lengthwise seal.

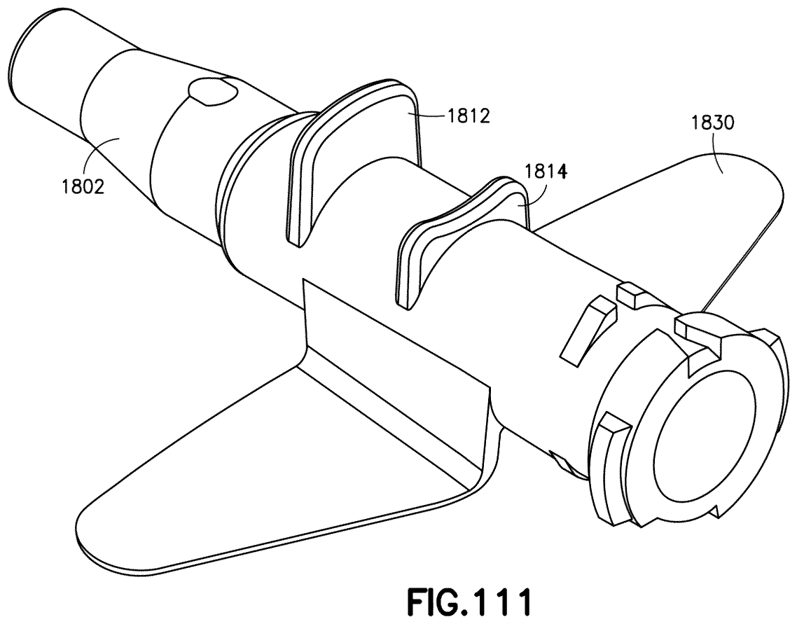

FIG. 109 illustrates three ribs 1714, 1716 and 1718; however, a single rib 1814 may be utilized to provide the necessary anti-rotational effect as illustrated in FIGS. 110 and 111. The rib(s) 1714, 1716, 1718 and 1814 should be a distance from the push tab 1712/1812 where the rib is far enough from the push tab 1712/1812 to contact a user's advancing finger pad to provide stability but not so close that the finger pad does not contact the rib. The catheter hub 1802 may be manufactured with or without wings 1830 as shown in FIGS. 110 and 111, respectively.

The foregoing detailed description of the certain exemplary embodiments has been provided for the purpose of explaining the principles of the invention and its practical application, thereby enabling others skilled in the art to understand the invention for various embodiments and with various modifications as are suited to the particular use contemplated. This description is not necessarily intended to be exhaustive or to limit the invention to the exemplary embodiments disclosed. Any of the embodiments and/or elements disclosed herein may be combined with one another to form various additional embodiments not specifically disclosed. Accordingly, additional embodiments are possible and are intended to be encompassed within this specification and the scope of the appended claims. The specification describes specific examples to accomplish a more general goal that may be accomplished in another way.

As used in this application, the terms “front,” “rear,” “upper,” “lower,” “upwardly,” “downwardly,” and other orientational descriptors are intended to facilitate the description of the exemplary embodiments of the present invention, and are not intended to limit the structure of the exemplary embodiments of the present invention to any particular position or orientation. Terms of degree, such as “substantially” or “approximately” are understood by those of ordinary skill to refer to reasonable ranges outside of the given value, for example, general tolerances associated with manufacturing, assembly, and use of the described embodiments.

What is claimed is:

1. A medical device, comprising:
a hub or housing including:
a push tab disposed on an upper surface of the hub or housing; and
a rib disposed on the upper surface of the hub or housing, the rib including a top surface and the rib being proximal to the push tab; and
a cannula directly or indirectly connected to the hub or housing, wherein
the top surface of the rib includes a cradle shape for resisting rotation of the hub or housing when contacted by a finger of a user.

2. The medical device of claim 1, wherein the push tab extends radially from the upper surface of the hub or housing at a lower portion of the push tab, to an upper portion of the push tab.

3. The medical device of claim 1, wherein the rib extends radially from the upper surface of the hub or housing at a lower portion of the rib, to the top surface of the rib.

4. The medical device of claim 1, wherein the push tab, the rib and the hub or housing are integrally formed as a unitary structure.

5. The medical device of claim 1, wherein the cradle shape includes a concave surface.

6. The medical device of claim 1, wherein the push tab and the rib are parallel to each other.

7. The medical device of claim 1, wherein a height of the push tab is greater than a height of the rib.

8. The medical device of claim 7, wherein
the height of the push tab is measured in a radial direction from the upper surface of the hub or housing to an upper portion of the push tab; and
the height of the rib is measured in a radial direction from the upper surface of the hub or housing to the top surface of the rib.

9. The medical device of claim 1, wherein
the cannula comprises a catheter tube of a catheter; and
the hub or housing comprises a catheter hub of the catheter.

10. The medical device of claim 1, wherein
the cannula comprises a catheter tube of a safety catheter; and
the hub or housing comprises a tip shield for an introducer needle of the safety catheter.

11. The medical device of claim 1, wherein the hub or housing includes two wings.

12. A medical device, comprising:
a hub or housing including:
a push tab disposed on an upper surface of the hub or housing; and
a plurality of ribs disposed on the upper surface of the hub or housing, the plurality of ribs each including a top surface and being proximal to the push tab; and
a cannula directly or indirectly connected to the hub or housing, wherein
the top surface of one of the plurality of ribs includes a cradle shape for resisting rotation of the hub or housing when contacted by a finger of a user.

13. The medical device of claim 12, wherein the plurality of ribs are all of a same height.

14. The medical device of claim 12, wherein all of the plurality of ribs includes the cradle shape.
